(19) United States
(12) Patent Application Publication
(10) Pub. No.: US 2023/0061342 A1
(43) Pub. Date: Mar. 2, 2023

(54) ANTI-MICROBIAL COATING

(71) Applicant: Royal Melbourne Institute of Technology, Melbourne, Victoria (AU)

(72) Inventors: Sumeet WALIA, Melbourne (AU); Aaron ELBOURNE, Melbourne (AU); Vi Khanh TRUONG, Melbourne (AU); Sruthi KURIAKOSE, Melbourne (AU)

(21) Appl. No.: 17/759,155

(22) PCT Filed: Jan. 20, 2021
(86) PCT No.: PCT/AU2021/050032

§ 371 Date: Jul. 20, 2022

(30) Foreign Application Priority Data
Jan. 20, 2020 (AU) 2020900142

(51) Int. Cl. A61L 27/32; A61L 27/54

(52) U.S. Cl. CPC A61L 27/32; A61L 27/54; A61L 2420/02; A61L 2300/404; A61L 2400/12

(57) ABSTRACT

An article comprising a substrate and an anti-microbial coating, wherein the anti-microbial coating comprises at least one solvent-free black phosphorus flake.

### TECHNICAL FIELD

The present invention relates to an anti-microbial coating and a method for applying said coating to a surface of a substrate.

The invention has been developed primarily for use in protecting articles from contamination by bacterial and/or fungal infection and will be described hereinafter with reference to this application.

The following discussion of the background to the invention is intended to facilitate an understanding of the invention. However, it should be appreciated that the discussion is not an acknowledgement or admission that any of the material referred to below, and anywhere else in the specification, was published, known or part of the common general knowledge in Australia or any other country as at the priority date of any one of the claims of this specification.

### BACKGROUND OF INVENTION

Microbe infections remain a significant medical concern, often with life-threatening consequences.¹⁻¹⁰ This situation has been exacerbated by the emergence of anti-microbial resistance (AMR), which is a direct result of the on-going misuse and over-prescription of anti-microbial agents.^{4, 6, 9-12} Indeed, resistant bacteria such as methicillin-resistant Staphylococcus aureus (MRSA) and carbapenem-resistant Enterobacteriaceae (CRE), which are unresponsive to most conventional antibiotics, have been widely reported.¹³⁻¹⁶ These factors have contributed to a situation where post-operative infections are rapidly increasing, while the last reliable preventative and therapeutic measures are beginning to fail.^{13,14, 17} Recent economic projections have estimated that AMR could be responsible for upwards of 10 million deaths per annum by 2050,¹⁸ equating to US$100 trillion in healthcare costs and reducing world economic output by around 2-3.5%, if new antibacterial therapies are not developed.^{18, 19} In tandem, fungal cells have been estimated to infect 1.7 billion people annually, resulting in approximately 1.5 million deaths per annum.^{20, 21} Unfortunately, deaths due to fungal infections are also increasing, with the mortality rate associated with some species often exceeding 50%, which further increases towards 100% if treatment is delayed.^{20, 21} Despite these figures, the contribution of fungal infections to the global burden of disease remains unrecognised. In Australia, over a five-year period fungal infections cost an estimated $583 million.²² The median cost for one invasive fungal disease (IFD) is AU$30,957, increasing to AU$80,291 if the patient is admitted to an intensive care unit.²³

In response, considerable scientific and medical research has focused on the development of surfaces that are capable of mitigating both bacterial and fungal growth and biofilm formation on surfaces.^{5, 24-26} Initial research has focussed on the addition of anti-microbial or inhibitory agents to the outer surface of biocompatible materials,^{27, 28} such as antibiotics,^{29, 30} and polymers,^{31, 32} among others.^{3, 29, 30, 33-40} However, due to several disadvantages, such as patient tissue sensitivity, increasing antibiotic resistance,³ toxicity concerns about nanomaterials,⁴¹ and dosage complications,^{3, 39} additive methodologies have become less viable as a long-term, anti-microbial solution. In response, both scientific and medical research has increasingly been focussed on the development of next-generation therapeutic measures, often with the view to target bacterial physiology previously not exploited in current antibiotic measures.⁵

Numerous avenues have been explored, including the study of nano-particles and nano-materials, as means for the preparation of new therapeutic measures.^{40, 42-29} More recently, two-dimensional and low-dimensional materials have received considerable attention as potential anti-microbial agents. These materials have included graphene,⁵⁰ graphene oxide,⁵⁰⁻⁵⁷ molybdenum disulphide,^{52, 58} and black phosphorus (BP),⁵⁹⁻⁶¹ in both their pure and composite forms. Of these low-dimensional materials, BP has emerged as a promising nanomaterial with biomedical applications, including drug delivery, biosensing, bioimaging, and as a promising antibacterial technology.⁶²⁻⁶⁵ Studies into the anti-microbial activity of BP, and BP-based composites, have solely focussed on the antibacterial properties of BP that has been liquid exfoliated using solvents. However, the antibacterial properties associated with the liquid exfoliants have not been fully explored to date, and thus the role of these solvent in the noted antibacterial properties cannot be ruled out. Further, solvent suspended black phosphorus cannot be used to impart anti-microbial properties to items.

In relation to the use of black phosphorous as an antibacterial agent Sun et al in Nanoscale, 2018, 10, 12543-12553 demonstrates the viability of BP in solution to act as an anti-bacterial agent. This paper teaches the solution exfoliation of black phosphorous leading to black phosphorous in solution. The solution is then stimulated by irradiation by exposure to 3 minutes of irradiation with near infrared. The requirement for stimulation in this way is undesirable as the irradiation can cause damage to healthy cells as well as the desired pathogen. In addition, the data demonstrates that in order to get above 80% death rate needed a concentration of 640 μg/mL (see FIG. 9) whereas at a concentration of 80 μg/mL there was only around a 20% death rate. As will be appreciated lower concentrations are desirable in order to reduce side effects.

In a similar way Xiong et al in Ecotoxicology and Environmental safety 161 (2018) 507-5014 also reports the solvent exfoliation of BP (using sonication) to produce solutions of BP in water. This study reported (see FIG. 2) that at a concentration of 10 μg/mL there was less than 20% dead, at 50 μg/mL gave around 80% dead whilst at 100 μg/mL there was a 90-100% death rate. Once again, these rates are still relatively high.

Ouyang et al in J. mater Chem B, 2018, 6, 6302-6310 reports the solution exfoliation of BP to produce different solutions of BP in basic N-methyl pyrollidone. As with Sun above the solution is then stimulated by irradiation by exposure to 5 minutes of irradiation with near infrared. The results demonstrated that at 38 mg/mL there was an approximate kill rate of 75%.

Finally, Li et all in ACS Appl. Nano mater. 2019, 2, 1202-1209 teaches the solvent based exfoliation of BP (using sonication) in NMP to form solutions of BP nanosheets in solution. The in-solution sheets were then modified with Titanium complexes and use as anti-bacterial agents. The reported data demonstrates that at a concentration of around 35 μg/ml lead to about 20% inhibition.

Accordingly, whilst BP in solution has been shown to be potentially effective as an anti-bacterial agent it relies on the solvent exfoliation of BP which leads to BP in solution. This is undesirable in some respects as there may be side effects bought about by the solvent and it is unclear whether the solvent is playing any part in the reported activity. In addition, the dosages required were relatively high due to solvent dilution effects.

The present invention seeks to provide an anti-microbial coating and a method for applying said coating to a surface of a substrate, which will overcome or substantially ameliorate at least some of the deficiencies of the prior art, or to at least provide an alternative. In particular we seek to provide a coating whereby the amount of black phosphorous is at lower concentrations than thought previously possible using solution based methods.

### SUMMARY OF INVENTION

According to a first aspect of the present invention, there is provided an article comprising a substrate and an anti-microbial coating, wherein the anti-microbial coating comprises at least one solvent-free black phosphorus flake. The applicants have found that by mechanical exfoliation of BP we can provide concentrations of black phosphorous on a surface significantly lower than that reported for solvent exfoliated systems. Indeed, a skilled worker in the art on reading the studies directed towards solution based black phosphorous as discussed above would not be lead to producing a black phosphorous coating at the levels provided in the present application.

In one embodiment the solvent free black phosphorous flake has been exfoliated. Exfoliation of black phosphorous flakes leads to the formation of either a mono or few layer black phosphorus flake. Black phosphorous flakes of this type are essentially “two-dimensional” as their thicknesses are approaching the atomic scale. In one embodiment the black phosphorous flake has from 1 to 5 layers. In one embodiment the black phosphorous flake has from 1 to 4 layers. In one embodiment the black phosphorous flake has from 1 to 3 layers. In one embodiment the black phosphorous flake is a monolayer black phosphorous flake. In one embodiment the black phosphorous flake has 2 layers. In one embodiment the black phosphorous flake has 3 layers.

Suitably, the at least one black phosphorus flake generates reactive oxygen species (ROS) that are active towards at least some types of micro-organisms.

In one embodiment, the anti-microbial coating prevents the growth of, or kills, micro-organisms selected from the group consisting of bacterial cells and fungal cells or spores.

In one embodiment, the anti-microbial coating has a density that falls within a range of from about 0.1 ng of black phosphorus per μm² of substrate to about 1.0 ng of black phosphorus per μm² of substrate.

In one embodiment, the anti-microbial coating has a density that falls within a range of from about 0.2 ng of black phosphorus per μm² of substrate to about 0.6 ng of black phosphorus per μm² of substrate

In one embodiment, the anti-microbial coating has a density that falls within a range of from about 0.4 ng of black phosphorus per μm² of substrate.

In one embodiment, the anti-microbial coating prevents the growth of, or kills, one or more bacterial species selected from the group consisting of: Escherichia coli, Pseudomonas aeruginosa, methicillin-resistant Staphylococcus aureus (MRSA) (resistive species), Salmonella typhimurium, and Bacillus cereus.

In one embodiment, the anti-microbial coating prevents the growth of, or kills, one or more fungal species selected from the group consisting of: Candida albicans, Candida auris, sensitive Cryptococcus neoformans, fluconazole-resistant Cryptococcus neoformans and Ampicillin-resistant Cryptococcus neoformans.

In one embodiment, the anti-microbial coating kills at least about 90% of microorganisms within a period of 120 minutes under ambient conditions, wherein the microorganism is selected from the group consisting of: methicillin-resistant Staphylococcus aureus, or Escherichia coli, or Pseudomonas aeruginosa, or Salmonella typhimurium, or Bacillus cereus, or Candida albicans, or Candida auris, or Sensitive Cryptococcus neoformans, or fluconazole-resistant Cryptococcus neoformans, or Ampicillin-resistant Cryptococcus neoformans.

In one embodiment, the at least one black phosphorus flake has an average thickness that falls within a range of from about 10 nm to about 120 nm

In one embodiment, the at least one black phosphorus flake has an average thickness that falls within a range of from about 15 nm to about 90 nm

In one embodiment, the at least one black phosphorus flake has an average lateral dimension that falls within a range of from about 500 nm to about 5 μm.

Preferably, the at least one black phosphorus flake is produced from a black phosphorus crystal by mechanical exfoliation.

Suitably, the mechanical exfoliation is conducted in a solvent-free environment.

In one embodiment, the at least one black phosphorus flake is physically adsorbed on to a surface of the substrate.

In one embodiment, the at least one black phosphorus flake is deposited onto a surface of the substrate by contacting the substrate surface with an applicator having at least one black phosphorus flake in contact with a surface thereof.

In one embodiment, the reactive oxygen species (ROS) are generated when the black phosphorus flake is exposed to atmospheric oxygen.

In one embodiment, the reactive oxygen species (ROS) are generated under ambient conditions.

Suitably, the reactive oxygen species are selected from the group consisting of a singlet oxygen radical (¹O₂), a hydroxy radical (OH.), a superoxide radical (O₂.⁻) and hydrogen peroxide (H₂O₂).

In one embodiment, the reactive oxygen species (ROS) are not substantially cytotoxic towards mammalian cells.

In one embodiment, the article is an implant, a medical instrument, a bioscaffold, or a woven or non-woven textile.

Preferably, the substrate is selected from the group consisting of: metal, an alloy, a polymer, a textile, a glass, a ceramic, or any combination thereof.

Preferably, the metal is selected from the group consisting of titanium, gold, stainless steel, aluminium, copper, or any combination thereof.

In one embodiment, the at least one black phosphorous flake is adhered to the substrate.

According to a second aspect of the present invention, there is provided a method of producing an article having an anti-microbial coating, comprising: providing a substrate; depositing at least one black phosphorus flake onto a surface of the substrate in the absence of solvent.

In one embodiment the solvent free black phosphorous flake has been exfoliated. Exfoliation of black phosphorous flakes leads to the formation of either a mono or few layer black phosphorus flake. Black phosphorous flakes of this type are essentially “two-dimensional” as their thicknesses are approaching the atomic scale. In one embodiment the black phosphorous flake has from 1 to 5 layers. In one embodiment the black phosphorous flake has from 1 to 4 layers. In one embodiment the black phosphorous flake has from 1 to 3 layers. In one embodiment the black phosphorous flake is a monolayer black phosphorous flake. In one embodiment the black phosphorous flake has 2 layers. In one embodiment the black phosphorous flake has 3 layers.

Suitably, the at least one black phosphorus flake generates reactive oxygen species (ROS) that are active towards at least some types of micro-organisms.

In one embodiment, the anti-microbial coating prevents the growth of, or kills, micro-organisms selected from the group consisting of bacterial cells, viruses [TBC] and fungal cells or spores.

In one embodiment, the anti-microbial coating has a density that falls within a range of from about 0.1 ng of black phosphorus per μm² of substrate to about 1.0 ng of black phosphorus per μm² of substrate

In one embodiment, the anti-microbial coating has a density that falls within a range of from about 0.2 ng of black phosphorus per μm² of substrate to about 0.6 ng of black phosphorus per μm² of substrate

In one embodiment, anti-microbial coating has a density of about 0.4 ng of black phosphorus per μm² of substrate.

In one embodiment, the anti-microbial coating prevents the growth of, or kills, one or more bacterial species selected from the group consisting of: Escherichia coli, Pseudomonas aeruginosa, methicillin-resistant Staphylococcus aureus (MRSA) (resistive species), Salmonella typhimurium or Bacillus cereus.

In one embodiment, the anti-microbial coating prevents the growth of, or kills, one or more fungal species selected from the group consisting of: Candida albicans, Candida auris, sensitive Cryptococcus neoformans, fluconazole-resistant Cryptococcus neoformans or Ampicillin-resistant Cryptococcus neoformans.

In one embodiment, the anti-microbial coating kills at least about 90% of microorganisms within a period of 120 minutes under ambient conditions, wherein the microorganism is selected from the group consisting of: methicillin-resistant Staphylococcus aureus, or Escherichia coli, or Pseudomonas aeruginosa, or Salmonella typhimurium, or Bacillus cereus, or Candida albicans, or Candida auris, or Sensitive Cryptococcus neoformans, or fluconazole-resistant Cryptococcus neoformans, or Ampicillin-resistant Cryptococcus neoformans.

In one embodiment, the at least one black phosphorus flake has an average thickness that falls within a range of from about 10 nm to about 120 nm

In one embodiment, the at least one black phosphorus flake has an average thickness that falls within a range of from about 15 nm to about 90 nm

In one embodiment, the at least one black phosphorus flake has an average lateral dimension that falls within a range of from about 500 nm to about 5 μm.

In one embodiment, the at least one black phosphorus flake is produced from a black phosphorus crystal by mechanical exfoliation.

Suitably, the mechanical exfoliation is conducted in a solvent-free environment.

In one embodiment, the at least one black phosphorus flake is physically adsorbed on to a surface of the substrate.

In one embodiment, the at least one black phosphorus flake is deposited onto a surface of the substrate by contacting the substrate surface with an applicator having at least one black phosphorus flake in contact with a surface thereof.

In one embodiment, the reactive oxygen species (ROS) are generated when the black phosphorus flake is exposed to atmospheric oxygen.

In one embodiment, the reactive oxygen species (ROS) are generated under ambient conditions.

Suitably, the reactive oxygen species are selected from the group consisting of a singlet oxygen radical (¹O₂), a hydroxy radical (OH.), a superoxide radical (O₂.⁻) and hydrogen peroxide (H₂O₂).

In one embodiment, the reactive oxygen species (ROS) are not substantially cytotoxic towards mammalian cells.

In one embodiment, the at least one black phosphorous flake is adhered to the substrate.

Preferably, the substrate is selected from the group consisting of: metal, an alloy, a polymer, a textile, a glass, a ceramic, or any combination thereof.

Preferably, the metal is selected from the group consisting of titanium, gold, stainless steel, aluminium, copper, or any combination thereof.

Preferably, the substrate is produced from a medical grade metal or alloy.

In one embodiment, the substrate is a surgical implant.

In one embodiment, the substrate is a bioscaffold.

In one embodiment, the substrate is a woven or non-woven textile.

According to a third aspect of the present invention, there is provided an article produced according to the method of the second aspect.

According to a fourth aspect of the present invention, there is provided a use of at least one solvent-free black phosphorus flake in the manufacture of an anti-microbial coating for a substrate.

According to a fifth aspect of the present invention, there is provided a solvent-free black phosphorous flake for use in an anti-microbial coating on a substrate.

According to a sixth aspect of the present invention, there is provided a method of producing at least one solvent-free black phosphorus flake with anti-microbial activity, comprising: contacting a black phosphorus crystal with an applicator having an adhesive surface to cause the applicator to adhere to a surface of the black phosphorus crystal; and removing the applicator from the surface of the black phosphorous crystal, thereby mechanically exfoliating at least one black phosphorous flake from the black phosphorous crystal.

According to a seventh aspect of the present invention, there is provided an anti-microbial coating, comprising: at least one black phosphorus flake produced in the absence of solvent.

Suitably, the at least one black phosphorus flake generates reactive oxygen species (ROS) that are active towards at least some types of micro-organisms.

According to an eighth aspect of the present invention, there is provided an article comprising a substrate and an anti-microbial coating according to the seventh aspect, wherein the at least one phosphorus flake is deposited on a surface of the substrate in the absence of solvent.

Other aspects of the invention are also disclosed.

### BRIEF DESCRIPTION OF DRAWINGS

Notwithstanding any other forms which may fall within the scope of the present invention, preferred embodiments of the invention will now be described, by way of example only, with reference to the accompanying drawings in which:

FIG. 1 shows: A), a schematic representation of the structure of the few-layer BP atomic lattice in which the grey balls represent phosphorus atoms, and the connecting lines between these atoms represent atomic bonds, B) an SEM image of a single few-layer BP flake, showing high quality sheets attached to a glass substrate following mechanical exfoliation, and C) EELS spectra of freshly exfoliated BP showing the i) P-L_{2,3} and L₁edge and the ii) O-K edge. Importantly, there is no peak present in the O-K region, which suggests minimal oxidation;

FIG. 2 shows A) a STEM image of a single BP flake, along with the corresponding chemical mapping spectra of B) phosphorus and C) oxygen, D) HR-TEM image of the mechanically exfoliated few-layer BP, with the insert showing the lattice spacing between atomic layers. E) an AFM image of several few-layer BP flakes, revealing the average lateral morphology and height of the flakes, in which the inset image to the left shows a selection of section profiles, which correspond to the lines over selected BP flakes in the image. These profiles show the relative height of a selection of few-layer BP flakes, F) a Raman spectrum of the mechanically exfoliated few-layer BP attained following deposition on a silicon substrate, And histograms showing G) the population of thickness and H) the lateral size ranges of the of few-layer BP following mechanical exfoliation;

FIG. 3 shows A) time-lapse confocal laser scanning microscopy (CLSM) images of MRSA and fluconazole resistant Cryptococcus neoformans (F) cells following exposure to BP, and B) a plot that shows the anti-microbial performance of MRSA and Cryptococcus neoformans (F) cells quantified as a percentage of dead cells as a function of time;

FIG. 4 shows A) cell viability of the microbial cells incubated in the presence of few-layer BP as quantified from the CLSM images (see FIG. 3A), and B) control and phosphorus treated CLSM images of the bacteria, E. coli, P. aeruginosa, MRSA, S. typhimurium, and B. cereus, as well as the fungus C. albicans, C. auris, C. neoformans, C. neoformans F^{R}, and C. neoformans A^{R};

FIG. 5 shows SEM micrographs of control and phosphorus treated bacterial cells (see FIG. 5A) and fungal cells (see FIG. 5B). The bacteria are E. coli, P. aeruginosa, MRSA, S. typhimurium, B. Cereus, and the fungal cells are C. albicans, C. auris, C. neoformans (Sensitive), C. neoformans F^{R}, and C. neoformans A^{R} [The inset image shows a bacterial spore of the B. cereus species];

FIG. 6 shows Cell viability of L929 and BJ-5TA fibroblasts after 48 hours. A) Control and B) Phosphorus treated CLSM images of the fibroblast cells. For the L929 fibroblast, the nucleus of the stained with DAPI and the actin filaments were stained with rhodamine-phalloidin after 48 hours exposure. For the BJ-5TA fibroblasts, the Calcein-AM stain binds to viable cells (green) and Ethidium homodimer stain bind to the DNA of non-viable cells (red). C) Cell viability of the fibroblast cells incubated in the presence of few-layer BP and control as quantified by an MTS assay. N=6 for the L929 cell testing and N=3 for the BJ-5TA cell testing.

FIG. 7. shows Antimicrobial activity of BP coated medically relevant surfaces. A) Control and Phosphorus treated CLSM images of MRSA and C. neoformans (F^{R}) cells following 2 hours of exposure on Ti, PDMS, PET and a fabric bandage. B) Corresponding number of non-viable cells of each microbial species following exposure to BP coated on each surface. C) AFM images of Ti, PDMS, PET and an optical microscope image of the bandage surface obtained prior to BP deposition.

FIG. 8 shows high-resolution AFM images of the few-layer BP A) before, B) after mild exposure to a microbial solution, and C) 2 hours of exposure to the microbial solution; and

FIG. 9 shows SEM images of the few-layer BP A) before and B) after exposure to a microbial solution.

FIG. 10 shows. A) CLSM images of MRSA and C. neoformans (F^{R}) after 48 hours incubation with BP nanoflakes exposure and B) the corresponding percentages of non-viable cells.

FIG. 11. Band structure and optimized geometry for A, D) pristine BP; B, E) Single-defect BP; C, F) Single-defect BP reacted with O₂. The black line and red lines correspond to spin-up and spin-down states, respectively. G) Singlet oxygen formation based on phosphite decomposition intermediates. H) Reduction of O₂ to superoxide by the defect BP surface. I) O-O distance as a function of time in AIMD simulations of superoxide formation.

FIG. 12 shows. Hemolysis of BP flakes A) The change in relative absorbance of the RBC solution at 576 nm and B) the UV absorbance spectra of the BP treated RBC compared to a negative control.

### DETAILED DESCRIPTION

It is to be understood that the following description is for the purpose of describing particular embodiments only and is not intended to be limiting with respect to the above description.

The present invention is predicated on the finding that few-layer BP flakes in their native state, being devoid of a solvent, exhibit broad-spectrum anti-microbial activity towards several types of micro-organisms, including bacteria such as, Escherichia coli (E. coli), Pseudomonas aeruginosa (P. aeruginosa), MRSA, Salmonella typhimurium (S. typhimurium), and Bacillus cereus (B. cereus), as well as a number of fungal cells or spores, including Candida albicans (C. albicans), Candida auris (C. auris) and wild strains of Cryptococcus neoformans (C. neoformans (Sensitive)), fluconazole-resistant C. neoformans (C. neoformans F^{R}), and amphotericin B-resistant C. neoformans (C. neoformans A^{R}) cells.

Microscopic studies were used to assess the underlying anti-microbial activity of these native few-layer BP flakes, revealing that reactive oxide species (ROS) formation was integral to the antibacterial mechanism. The inventors have also surprisingly found that mammalian cells (such as the mouse L929 fibroblasts cell line) are substantially unaffected by the same production of reactive oxide species (ROS), with the cells thriving when exposed to identical BP concentrations. Without wanting to be bound by theory, this disparity between the ability of microorganisms and mammalian cells to deal with ROS likely depends on the fact that mammalian cells have improved mechanisms for sequestering ROS and repairing cellular damage induced by ROS. On the other hand, microorganisms, while having limited ability to cope with oxidative stress, often have a more rapid accumulation of cellular damage and therefore are more vulnerable to oxidative damage.

The utility of BP as a surface coating material was also studied for its effectiveness via surface functionalisation techniques. This study for the first time shows efficacy on a medical grade titanium implant opening a pathway to deploy this self-degradable BP at extremely low concentrations to kill a wide-range of pathogenic micro-organisms including resistant species. These few-layer thick coatings are amongst the thinnest anti-microbial coatings to eliminate the current issue of anti-microbial resistance crisis, where only nano-gram quantities of this material will be required.

What follows is a detailed description of the method required to prepare few-layer black phosphorus flakes for use as an anti-microbial coating on a wide range of suitable articles that find useful application in areas, including but not limited to, the medical device industry and healthcare in general.

Results

2.1 Characterization of Few-Layer Black Phosphorus

Black Phosphorus (BP) is a two-dimensional allotrope of phosphorus, with a characteristic non-planar, ridged lattice. As used herein the terms “few-layer BP” or “black phosphorous flakes” refers to the low-dimensional versions of the material, which consists of several to a few-tens of stacked BP sheets held together by weak van der Waal forces.

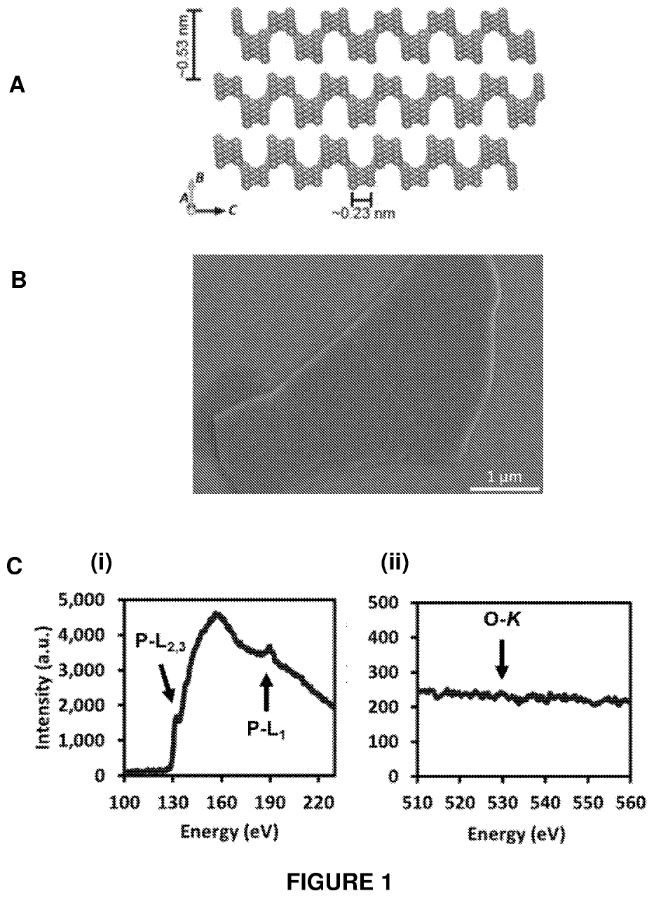

A schematic of the materials atomic lattice of BP is shown in FIG. 1A, including the lattice parameters. BP flakes were prepared for this study by contacting a bulk BP crystal (Smart elements) in its native state with an applicator having an adhesive surface, which in this instance is provided in the form of a blue polyvinyl chloride tape (Nitto), to cause the applicator to adhere to a surface of the bulk BP crystal, and then removing the applicator from the surface of the bulk BP crystal, thereby causing few-layer BP flakes to be peeled away from the bulk BP crystal surface by mechanical exfoliation.

It is important to stress here that the inventors surprisingly found that it was possible to prepare these few-layer BP flakes in the absence of solvent. That is, the few-layer BP flakes were not required to be solvent exfoliated, as is often reported in the literature, but rather mechanically exfoliated in the absence of solvent.

The inventors observed that it was possible to produce solvent-free BP flakes having an average thickness that falls within a broad range of from about 10 nm to about 5 μm.

In various examples, the inventors obtained solvent-free BP flakes with thicknesses of within a range of from about 10 nm to about 120 nm, and some within a range of from about 15 nm to about 90 nm.

The tape comprising the solvent-free BP flakes was then placed against the surface of a substrate of interest, and at least one of the BP flakes is touch transferred onto the desired surface.

In one embodiment, it was possible to deposit these solvent-free BP flakes onto the substrate surface via physical adsorption by relying on the native surface-surface interactions forces, without the need to apply an additional fixative.

In other embodiments, it is possible to secure the solvent-free BP flake(s) to the substrate surface with the aid of an additional fixative (not shown).

FIG. 1B shows an SEM image of a single BP flake, showing high quality sheets attached to the surface of a glass substrate following mechanical exfoliation.

FIG. 1C shows electron energy loss spectroscopy (EELS) spectra of freshly exfoliated solvent-free BP flakes showing the i) P-L_{2,3} and L₁edge and the ii) O-K edge. Importantly, there is no peak present in the O-K region, which suggests minimal oxidation.

Critically, BP produced in this fashion is deposited in a concentration density of from about 0.1 ng of black phosphorus per μm² of substrate to about 1.0 ng of black phosphorus per μm² of substrate, more preferably from about 0.2 ng of black phosphorus per μm² of substrate to about 0.6 ng of black phosphorus per μm² of substrate, with good results being obtained when the concentration density is around 0.4 ng/μm of substrate.⁶⁶ This concentration is significantly lower than that used when solvent suspended, or solvent stabilised, BP is analysed. Surprisingly, and despite this low concentration, the BP coating was found to have extremely high anti-microbial properties.

The term “concentration density” or “density” as used herein is used to describe how closely the coating molecules are packed on the surface of the substrate. Accordingly, as used herein the terms means the amount of black phosphorous per unit area (eg., cm²) applied to the substrate.

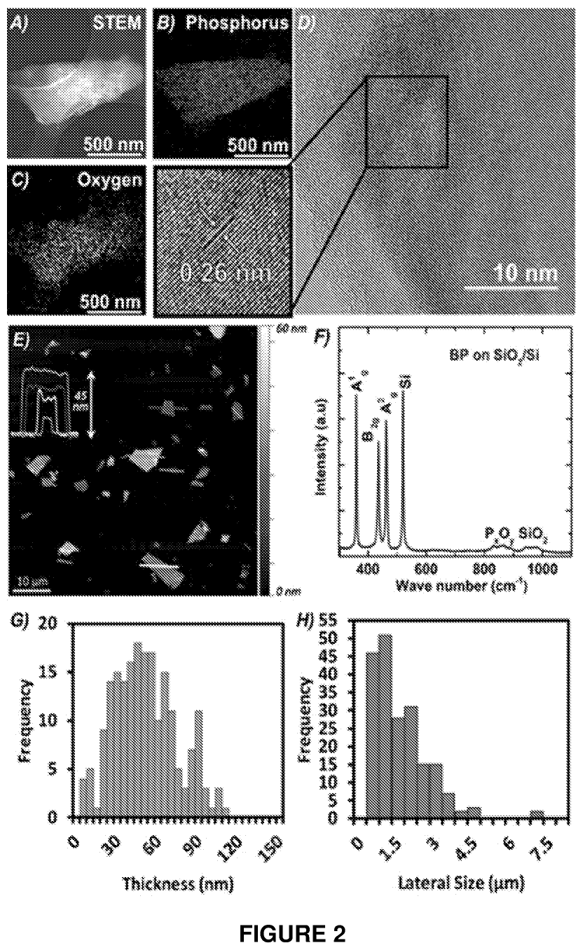

FIG. 2A shows a scanning transmission electron microscopy (STEM) micrograph of a single few-layer BP nanoflake following exposure to ambient conditions. During image acquisition, electron energy loss spectrometry (EELS) was used to chemically analyse the samples. Mapping the energy peaks associated with phosphorus and oxygen (see FIGS. 2B and 2C) reveals the distinct presence of elemental phosphorus and oxygen.

Analysis of the EDX spectra reveals commensurate data, showing that the flake is predominantly phosphorous, with a presence of oxygen species (see FIGS. 2B and 2C) which inherently forms on the surface. Oxygen is known to adsorb on the BP surface, which can oxidize further on exposure to ambient conditions.^{67, 6867} A high-resolution transmission electron microscope (HR-TEM) (FIG. 1D) was taken of deposited BP, and the insert shows a lattice spacing was found to be 0.26 nm,⁶⁸ which is consistent with the spacing between two sheets of phosphorus.

Raman spectroscopic analysis of the BP flakes following mechanical exfoliation was conducted. FIG. 2F shows the resulting Raman spectra revealing the three signature peaks of BP—the A¹_{g} (361 cm⁻¹), B²_{g}(438 cm⁻¹) and the A²_{g}(465 cm⁻¹);^{69, 70} the A¹_{g} modes originate from the out-of-plane vibrations of phosphorus atoms along the c-axis (see FIG. 1A), while the B²_{g} and A²_{g} modes arise from the in-plane vibrations of phosphorus atoms along the b-axis (armchair) and a-axis (zigzag), respectively (See FIG. 1A). The integrated Raman intensity ratio of A²_{g} to A¹_{g} is known to increase monotonically with decreasing layer numbers;⁷¹ however, atomic force microscopy (AFM) can more readily assess the variation of sizes within a heterogeneous population of BP flakes. As such, AFM imaging was employed to assess the morphology of the mechanically exfoliated BP flakes.

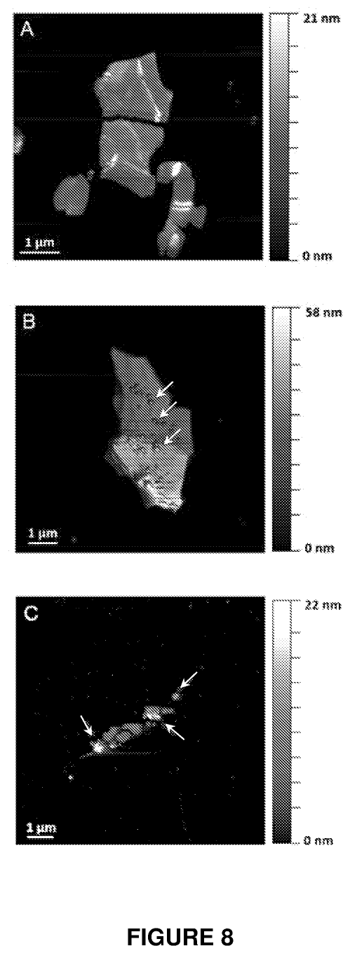

Representative AFM height profiles for the different thicknesses of BP flake obtained by mechanically exfoliation are shown in FIG. 2E. The mechanical exfoliation process was found to generate a distribution of few-layer BP flakes, with average lateral dimensions of 500 nm-5 μm and a somewhat stochastic range of thicknesses; in general, the highest proportion of flakes possess thickness in the range of 15-90 nm,⁷⁰ with a smaller proportion of flakes being less than 10 nm in thickness (see FIGS. 2G and 2H).⁷⁰ Higher resolution AFM images of several BP flakes are shown in FIG. 8.

2.2. Kinetics of the Anti-Microbial Activity of BP Flakes as a Surface Coating

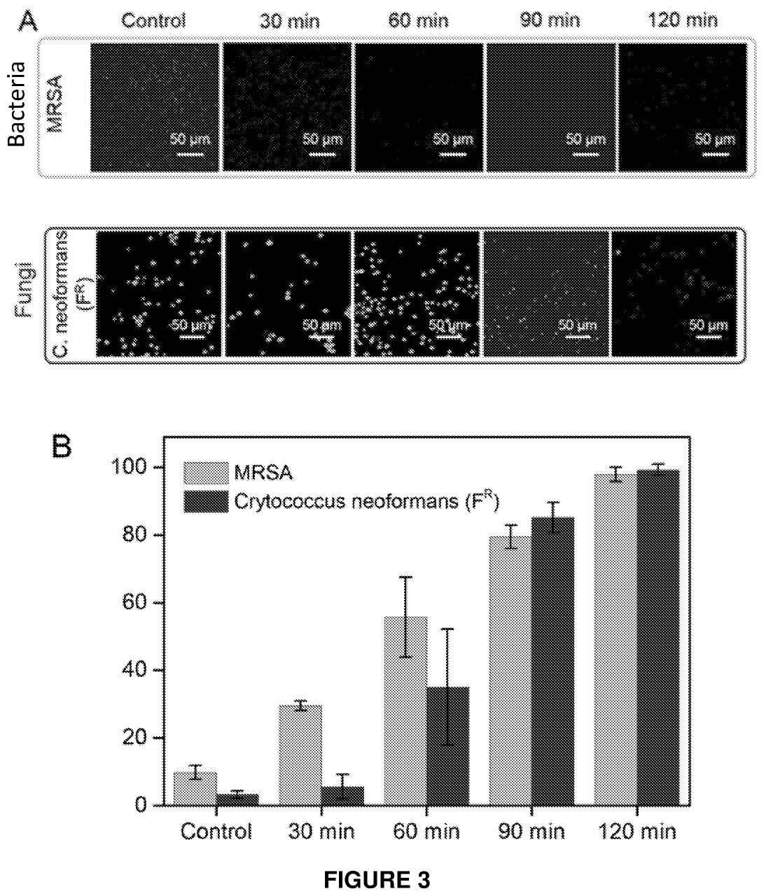

The anti-microbial effect of the BP flakes was assessed as a function of time using CLSM imaging. Two drug resistant microbes, specifically MRSA and C. neoformans F^{R}, were chosen as representative species to probe the time-correlated anti-microbial behaviour of the BP. Initially, control images were obtained for the untreated cells (see FIG. 3A) to provide a base-line of the natural viability of each microbe in solution. Subsequently, the same microbial solution was exposed to glass coverslip surfaces that had been coated with few-layer BP. The respective viability of the systems were assessed in situ at 30 minute time intervals over a period of 2.5 hours.

The relative proportion of viable and non-viable cells (live vs. dead) were then assessed at each time interval via a differential staining technique, where intact and damaged cells (or viable and dead cells respectively) were stained with SYTO® 9 (green colour when assessed via CLSM) and propidium iodide (PI, red colour in when assessed via CLSM), respectively. Specifically, SYTO® 9 fluorescent dye permeates both intact (viable) and damaged cell membranes and binds with nucleic acid.⁷² PI dyes only permeate through damaged (non-viable) cell membranes and bind in higher affinity with nucleic acids to replace SYTO® 9.⁷² This allows the visual differentiation between viable and non-viable cells, with the earlier fluorescing green when analysed via CLSM and the latter fluorescing red.

Representative CLSM images for each cell type, following treatment for the indicated time, are shown in FIG. 3A.

TABLE 1
Percentage of non-viable cells from FIG. 3 as a function of time

| Time | MRSA | C. neoformans F^{R} |
|---|---|---|
| Control (0 min) | 9.9% ± 2.1% | 3.2% ± 1.1% |

| | | | |
|---|---|---|---|
| 30 | min | 29.5% ± 1.45% | 5.5% ± 3.6% |
| 60 | min | 55.7% ± 11.9% | 35.0% ± 17% |
| 90 | min | 79.5% ± 3.4% | 85.1% ± 4.5% |
| 120 | min | 97.9% ± 2.2% | 99.3% ± 1.6% |

The relative numbers of live and dead cells were then determined and displayed as a percentage in the bar chart presented in FIG. 3B and in Table 1 above for both the control and treated cells, along with the associated standard deviation of cell viability. For MRSA and C. neoformans F^{R}, the average proportion of non-viable (dead) cells for the control samples were 9.8% and 3.2%, respectively.

Following exposure to the BP coated surfaces, the proportion of non-viable cells was observed to increase with time. Quantitatively, this equated to 29.5%, 55.7%, 79.5%, and 97.9% and 5.5%, 35.0%, 85.1%, and 99.3% of non-viable cells for the MRSA and C. neoformans F^{R} at time intervals of 30, 60, 90, and 120 min, respectively (see Table 1). Importantly, these values were observed to plateau at 120 min of exposure, with no statistically significant increase in anti-microbial activity noted beyond this time point. It is worthy to note that this result highlights a nominal exposure time of approximately 2 hours to achieve maximum anti-microbial activity of approximately 0.4 ng/μm² of BP material. Importantly, as briefly discussed above, the degree and rate of anti-microbial effect is significantly greater that that observed in liquid exfoliated BP flakes. While the precise reason for this disparity has not been determined (to date) it is likely that in the absence of solvent stabilisation, the BP flakes readily react with the atmosphere under ambient condition leading to a significant increase in ROS production and a resultantly higher anti-microbial activity.

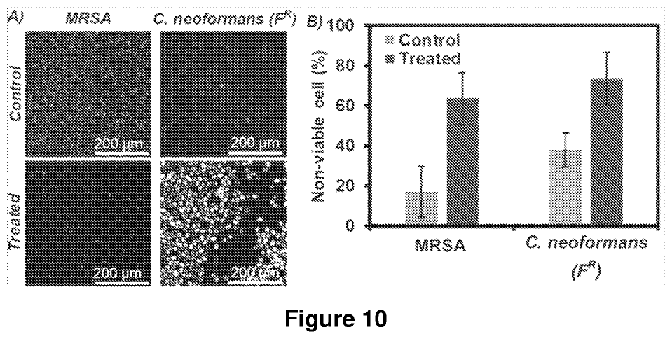

To further access the antimicrobial effectiveness of the deposited BP nanoflakes, the microbial solutions of MRSA and C. neoformans (F^{R}) were exposed to BP nanoflakes and incubated for 48 hours. The CLSM images of the resulting biofilm and the respective percent viability are shown in FIG. 10. In the high-resolution CLMS and AFM images, there is noticeable membrane deformation, with the size of the cells also decreasing. It was also notable that the mechanically exfoliated BP nanoflakes were able to inhibit the growth of MRSA and C. neoformans (F^{R}) over 48 hours. The average proportion of non-viable cells in the MRSA and C. neoformans (F^{R}) biofilms were 17.3±12% and 38.1%±8.5% respectively. After 48 hours of BP nanoflake exposure, the average proportion of non-viable cells was 63.9%±12.7% for MRSA and 73.3%±13.5% for C. neoformans (F^{R}) biofilms.

Further, this rapid degradation of solvent-free BP flakes provides a further advantage. As the coating degrades over a relatively short period of time, the risk of microorganisms becoming resistant decreases as a function of reduced exposure, and therefore reduced time for adaptation or selection for resistance. This is a feature not shared by the majority of anti-microbial coating currently used and reported in the art.

2.3 Anti-Microbial Activity of Solvent-Free Black Phosphorus

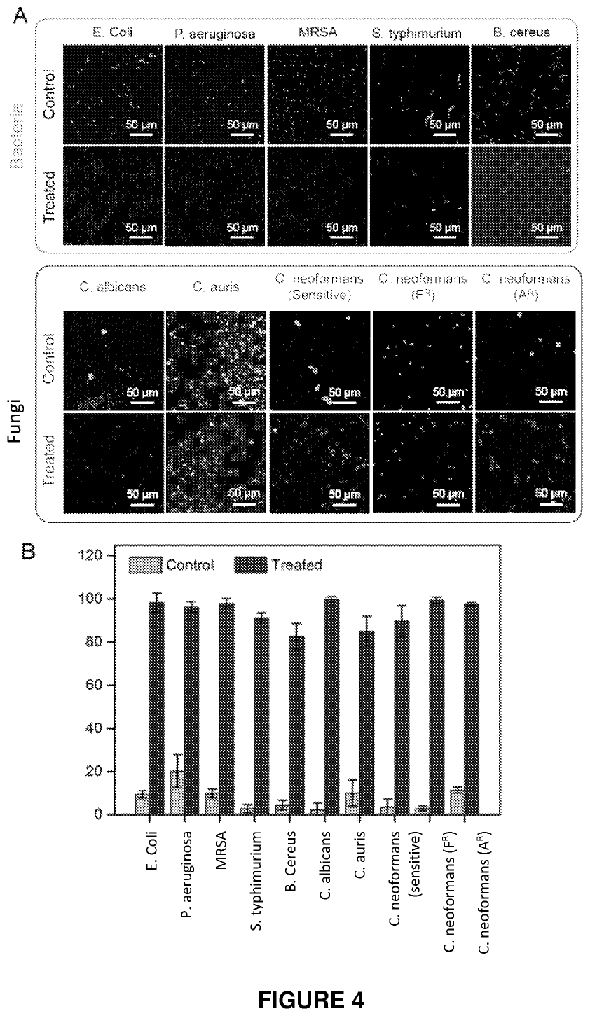

Following the initial time-lapse investigation of the solvent-free BP flakes against MRSA and C. neoformans F^{R} cells, the anti-microbial activity of the BP flakes was assessed against a broad-spectrum of pathogenic microbial species, including the bacteria, E. coli, P. aeruginosa, MRSA, S. typhimurium, and B. cereus, as well as the fungus C. albicans, C. auris, C. neoformans, C. neoformans F^{R}, and C. neoformans A^{R} using CSLM, following 2 hours of exposure to the material. Here, the relative proportion of live and dead cells was again visualized via fluorescent staining in the CSLM images (see FIG. 4A).

For the control and treated samples, the relative numbers of live and dead cells were then determined and displayed as a percentage in the bar chart presented in FIG. 3B. Specifically, these values are shown in Table 2 (below) for treated and control samples for E. coli, P. aeruginosa, MRSA, S. typhimurium, and B. cereus, as well as the fungus C. albicans, C. auris, C. neoformans, C. neoformans F^{R}, and C. neoformans A^{R}, respectively. The associated standard deviation of bactericidal activity is indicated by the error bars for each respective system investigated (see FIG. 4B).

2.4 Assessing the Anti-Microbial Mechanism of Solvent-Free Black Phosphorus

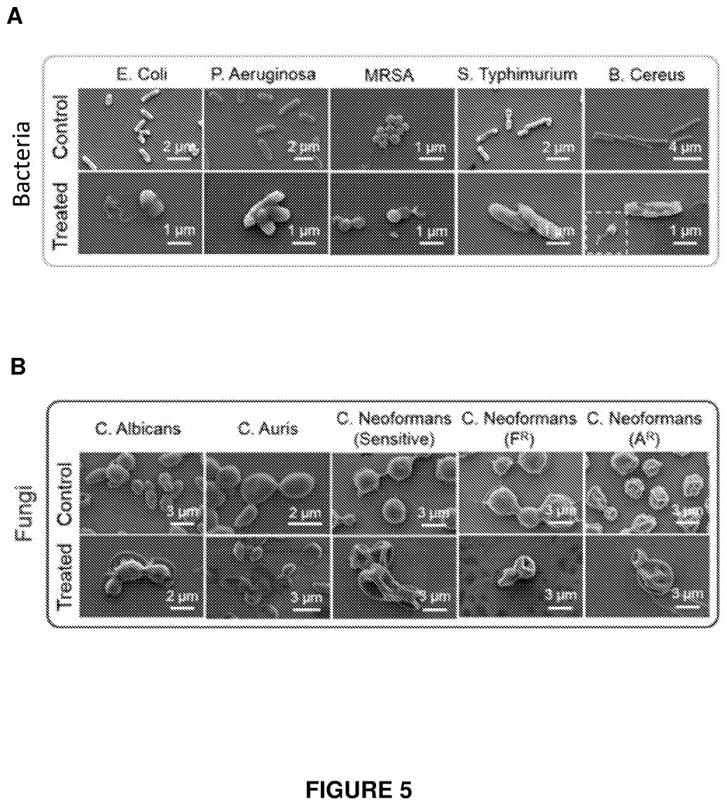

SEM imaging was employed to visualise the microbial species in their native state and following cellular-nanomaterial interactions. Control SEM micrographs of each microbial species investigated (top row) in the absence of BP adsorbed to a flat silicon surface are presented in FIG. 5. Here, both the bacterial (see FIG. 5A) and fungal (see FIG. 5B) species appear as they would in their native, state, with smooth, continuous exteriors. Importantly, the cells are visually intact and undamaged.

Following treatment with BP (see FIG. 5A, FIG. 5B, bottom rows) the cells within the SEM micrographs of all microbial species investigated appear markedly different. Closer, high-magnification inspection revealed that cells were visibly damaged and deflated, with all cells investigated bearing little resemblance to their control counter parts (see FIG. 5A, FIG. 5B, top-row). B. cereus is known to forms spores which are notoriously resistant due to the ultra-thick coat comprised of numerous peptidoglycan layers, which are present in these systems (see FIG. 5A).

Following treatment, the bacterial spores also appear damaged (see insets to respective species). Importantly, this reveals that BP-microbial interactions induce cellular defects, which aide to explain the high-number of inactivated cells quantified via CLSM investigation (see FIG. 4).

Without being bound by any one particular theory, the inventors believe that this activity towards at least some types of micro-organisms could be attributed to the ability of BP to interact with atmospheric oxygen under ambient conditions and produce reactive oxygen species (ROS). These ROS can comprise of several radicals such as singlet oxygen radicals (¹O₂), hydroxy radicals (OH.), superoxide radicals (O₂.⁻) and hydrogen peroxide (H₂O₂). to name a few.^{66, 73-75}

TABLE 2
Quantification of non-viable cells from FIG. 4.

| Species | Treated | Control |
|---|---|---|
| Escherichia coli | 98.3% ± 4.3% | 9.4% ± 1.7% |
| Pseudomonas aeruginosa | 96.2% ± 2.4% | 20.2% ± 7.3% |
| MRSA | 97.9% ± 2.2% | 9.8% ± 2.0% |
| Salmonella typhimurium | 91.2% ± 2.6% | 2.8% ± 1.8% |
| Bacillus cereus | 82.5% ± 6.2% | 4.4% ± 2.2% |
| Candida albicans | 99.9% ± 1.1% | 2.2% ± 3.3% |
| Candida auris | 85.2% ± 7.0% | 10.0% ± 6.0% |
| Cryptococcus neoformans (Sensitive) | 89.6% ± 7.2% | 3.5% ± 3.6% |
| Cryptococcus neoformans F^{R} | 99.3% ± 1.6% | 2.9% ± 1.1% |
| Cryptococcus neoformans A^{R} | 97.5% ± 0.8% | 11.4% ± 1.4% |

2.4.1 Computational Investigation of the Mechanism of ROS Generation

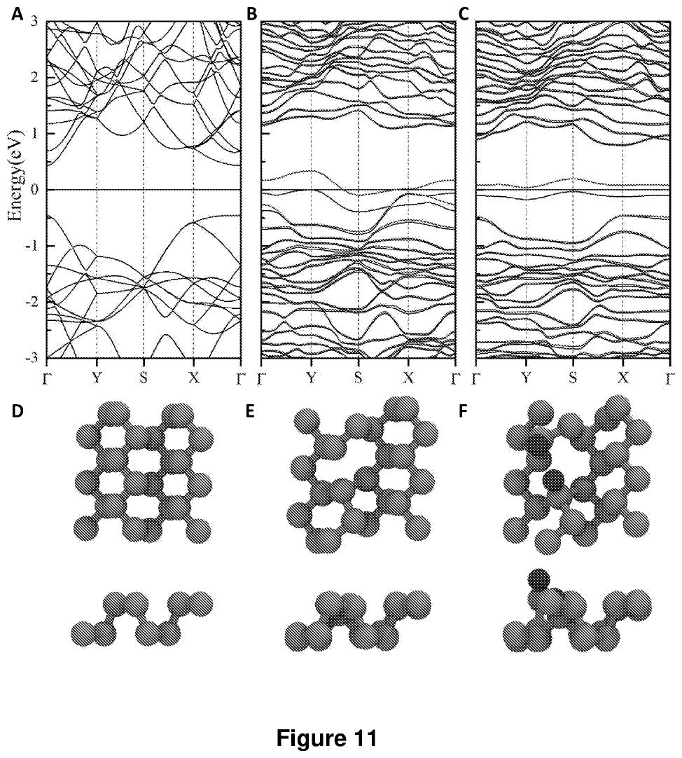

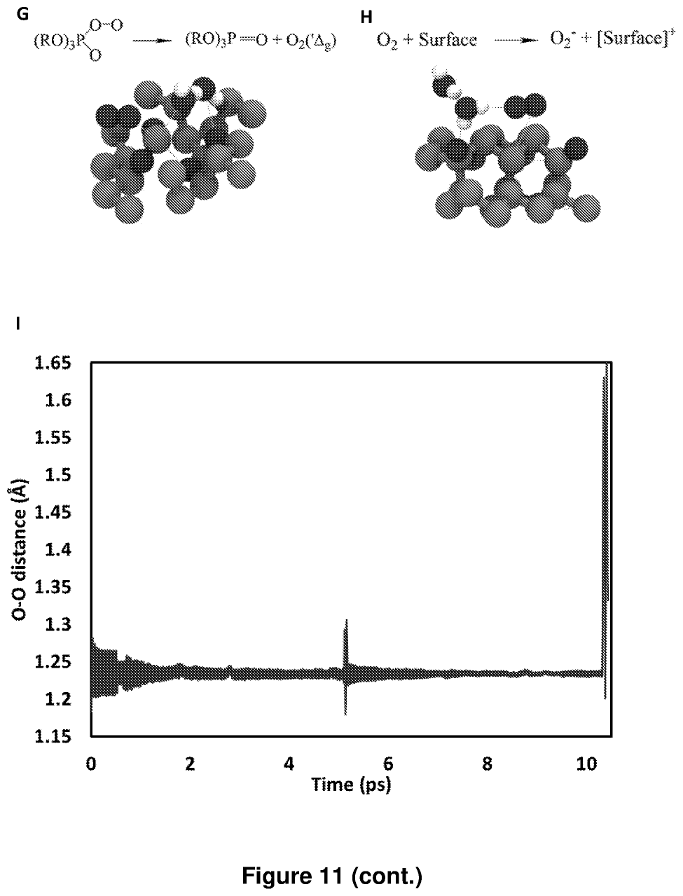

In order to gain a better understanding of ROS generation by few-layer BP, quantum chemical (QC) calculations exploring possible mechanisms of superoxide and singlet oxygen formation were performed. Previous studies have shown how superoxide radicals can be generated from pristine BP via a light-activation mechanism.⁷⁶ However, ROS measurements in this study demonstrate that superoxide radicals and singlet oxygen can be generated from few-layer BP in the absence of light. Optimized geometries and band structures of pristine BP, single-defect BP, and single-defect BP reacted with O₂ are shown in FIG. 11.

Clearly, the presence of an oxygen vacancy or an oxidized defect widens the band gap of the pristine system and introduces defect states. For a vacancy the gap transitions from being direct to indirect, while for the reacted oxygen system the gap remains direct but shifts to the X-point. For both defect systems, a new spin polarized state is introduced within the band gap region which intersects the Fermi level, indicating a partially filled state. This suggests that the system is reactive and likely to interact with other species if present. The presence of unpaired electrons induces a small magnetic moment on the system, which is localized on the atoms surrounding the defect, and explains the splitting of these bands in the band structures. Structurally, significant differences are observed.

At the oxidized defect site, the distance between adjacent non-bonded P atoms decreases from 3.3 Å to 3.0 Å (FIG. 11H). In the presence of water, the bond length of the O₂ molecule within ˜4.5 Å of this site lengthens during the AIMD simulation from 1.23 Å to 1.42 Å, which is indicative of superoxide/peroxide formation (FIG. 11).⁷⁷ For singlet oxygen, it is known that it can be generated during the decomposition of phosphites.⁷⁸ Analogous intermediates were shown to be formed on partially oxidized BP surfaces (FIG. 11G). Taken together, these results suggest that superoxide is formed directly by reduction of O₂ by the defect BP surface, while singlet oxygen can be formed as part of the degradation pathway of the oxidized surface.

2.5. Cytotoxicity of BP Flakes

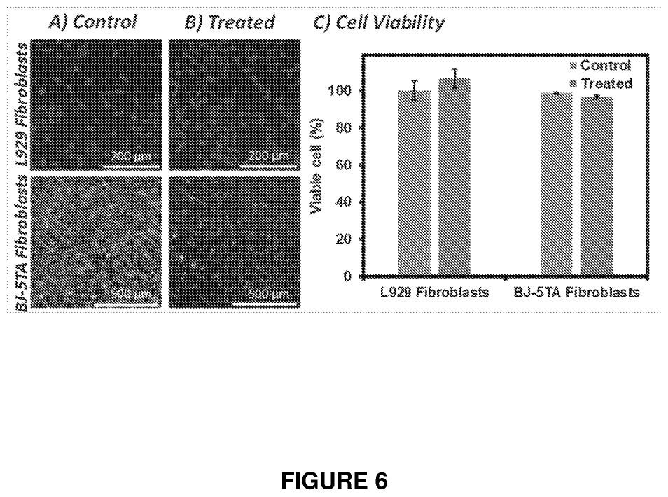

To assess the cytotoxicity of the BP flakes, the same concentration of BP flakes on surfaces was tested against L929 mouse fibroblasts following a 48-hour exposure to the material (see FIG. 6A). Following this, immortalised BJ-5TA human fibroblasts cells were also tested to provide greater insight in overall cytotoxicity. Two assays were used to assess the cell cytotoxicity: (1) fluorescent stains indicate the proliferation and spreading behaviour of fibroblast cells; (2) MTS methods (i.e. colorimetric indicator) of viability quantification. For the L929 fibroblasts, the viability was assessed via the reduction of a tetrazolium salt by viable cells to a coloured formazan product. The viability of the BJ-5TA fibroblasts was determined using the hydrolysation of calcein in viable cells and the binding of non-permeable ethidium homodimer dye to the DNA of non-viable cells. In FIGS. 6A and B, both L929 and BJ-5TA fibroblast cells were observed to have a negligible change in cell viability after 48 hours (FIG. 6C). The MTS assay confirmed that there is no observed cytotoxic effect of BP materials against the fibroblast cells (FIG. 6C). This is important, as the same density of BP flakes on surfaces can eradicate the majority of microbial pathogens (see FIG. 3 and FIG. 4), while having no discernible physiological effect on the growth of mammalian cells.

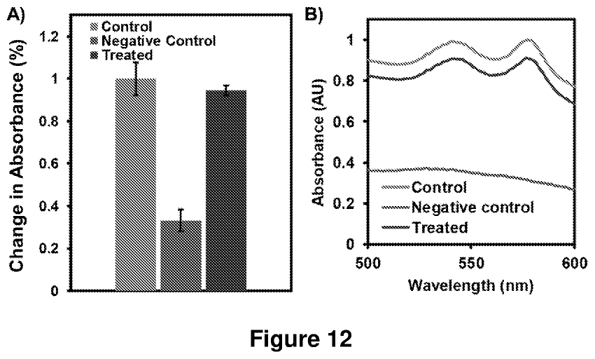

To this end, the inventors believe that the reactive oxygen species (ROS) generated as a result of photo-oxidation of the solvent-free BP flakes are not substantially cytotoxic towards mammalian cells. The hemocompatibility was also tested using ˜900 ng against red blood cells (RBC) with the resulting data shown in FIG. 12. Critically, the BP flakes did not induce hemolysis.⁷⁹

2.6. Assessing the Utility of BP Flakes as an Additive Anti-Microbial in a Practical Application

The previously described experiments were conducted on sterilised glass cover-slips. However, such substrates possess little utility beyond the laboratory. As such, few-layer BP (˜0.4 ng/μm²) was deposited on an industrial relevant substrate, specifically commercial pure grade 2 titanium (ASTM) discs, polyethylene terephthalate (PET) sheets, polydimethylsiloxane (PDMS) sheets and a commercially available fabric bandage. The Ti discs had a nominal diameter of 10 mm, and the PET, PDMS and bandage had a nominal size of 1 cm². The Ti surfaces are widely used in biomedical implants, both PET and PDMS are medically relevant substrates, and the bandages are used as common wound dressings. BP flakes were then mechanically exfoliated on the different substrates, as previously described, under cleanroom conditions to maintain sterile conditions.

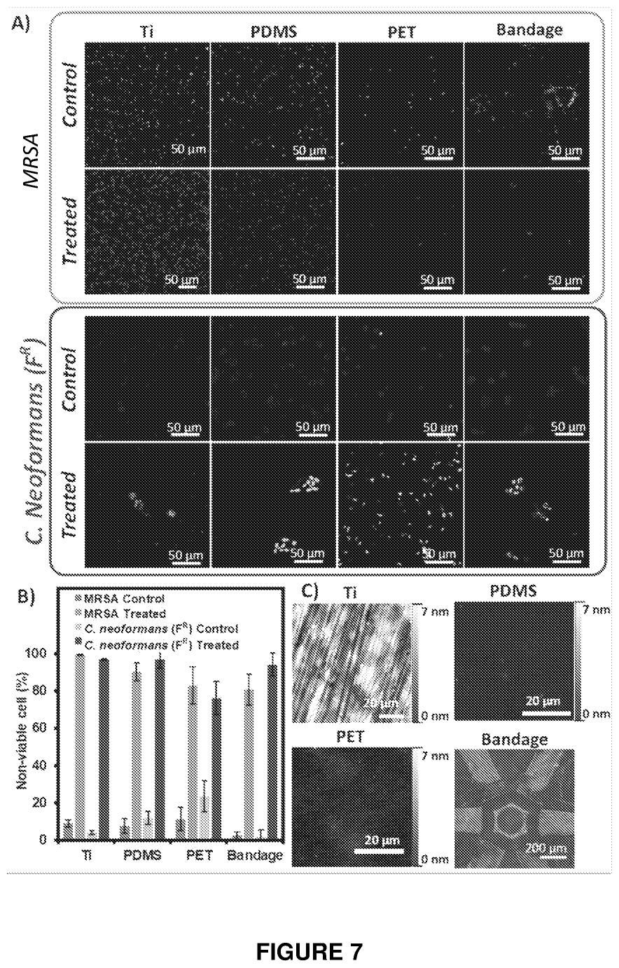

The antimicrobial effect of the few-layer BP coated surfaces was then assessed using CLSM images following a 2-hour exposure of the two previously chosen resistant microbes, specifically MRSA and C. neoformans (F^{R}) (see FIG. 7A). (see FIG. 7A). Viable cells were identified as green when analysed via CLSM and non-viable cells were identified as red via CLSM

Again, control systems were investigated for each as-received surface after 2 hours to assess any inherent antimicrobial activity. The proportion of non-viable cells were commensurate with those observed using the glass substrates for MRSA and C. neoformans (F^{R}), with Ti having 8.9% and 4.2%, PET having 8.9% and 4.2%, PDMS having 8.9% and 4.2%, and the bandage having 8.9% and 4.2% respectively. This shows that the as-received surfaces have no inherent antimicrobial activity.

Importantly, once coated with BP material, the Ti surfaces were found to be highly antimicrobial, with 99.6% and 97.0% of MRSA and C. neoformans (F^{R}) found to be non-viable following 2 hours of surface exposure. Similarly, PDMS had 90.5% of MRSA and 96.9% of C. neoformans (F^{R}) non-viable cells. The bandage was also highly effective with 80.8% of MRSA, and 94.3% of C. neoformans (F^{R}) cells, which were non-viable after 2 hours. This is an important finding, as it indicated that few-layer BP (˜0.4 ng/μm²) could be used as a coating's material to medically relevant substrates to achieve the high antimicrobial activity. For the treated PET surface, the percentage of non-viable cells were 83.0% of MRSA and 76.1% of C. neoformans (F^{R}). This lower percentage can be attributed to the lower amount of mechanically exfoliated BP deposited into the PET surface.

FIG. 7C shows 100 μm×100 μm AFM image obtained prior to BP deposition for Ti, PET and PDMS, and an optical microscope image of the bandage surface.

Discussion

Nanotechnology, such as the application of nanoparticles, low-dimensional materials, and surface coatings, have emerged as being applicable as next-generation anti-microbials technologies.^{5, 24, 25, 76} The rationale behind such studies has been to investigate alternative therapies to conventional treatment methods, such as antibiotics. Very recently, a few studies have noted the antibacterial properties of BP-based solutions^{59, 61, 77, 78} and its composite materials.^{60, 61}

The culmination of this work suggests that BP in solution in μg/mL concentration regimes can be highly antibacterial;^{59-61, 77, 78} many of these studies, however, also employed additional UV exposure to increase the inherent bactericidal activity,⁵⁹ while only exploring the efficacy of the material towards a few bacterial species. This information is summarized in Table 3, along with other commonly investigated antibacterial nanomaterials for comparison to the efficacy of BP elucidated in this study. More importantly, these studies all indicate that the primary antibacterial mechanism is the creation of, and interaction of the microbes with, reactive oxide species (ROS).^{67, 70, 74, 79}

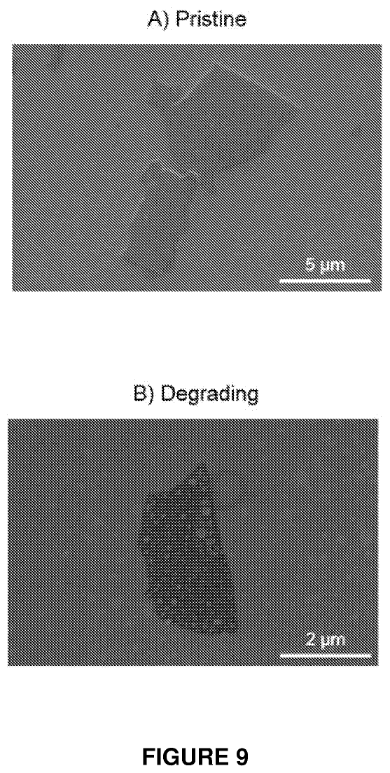

BP-based nanomaterials, such as nanoflakes and composites, are capable of producing ROS. This occurs as the material degrades under ambient conditions in air and in solution.^{66, 73-75, 80} AFM images of this process are shown in FIG. 8, while SEM images are shown in FIG. 9. There are two competing processes by which oxygen reacts with layered black phosphorus: edge degradation and surface degradation. The degradation process of BP flakes is multi-dimensional, with the edge degradation occurring faster than that at the surface.⁸¹

Specifically, BP-based material will produce significant quantities of .OH, .O₂⁻, and H₂O₂, where the precise concentration of ROS increases as a function of time and the initial base material concentration.^{66, 67, 70, 73-75, 79, 80} For microbes, such as bacterial and fungal cells, these ROS are capable of inducing cell lysis, causing cell membrane and intercellular damage.

In general, the anti-microbial activity of BP is thought to occur via two main pathways: 1) membrane disruption, following nanomaterial-microbial interaction, which degrades the cell integrity, or 2) by inducing the production of reactive oxide species (ROS) which initiates cell oxidation, oxidative stress, and ultimately cell lysis.

The results of this study are commensurate with this observation, as the microbial cells show significant membrane damage (see FIG. 5) and were largely non-viable following 2 hours of exposure to the BP nanoflakes (c.f. FIGS. 3, 4 and 7).

Previously,⁷³ the production of singlet oxygen species (¹O₂), as well as hydroxyl (OH.) and superoxide (O₂.⁻) radicals species which are produced by solvent-based mechanically exfoliated BP during degradation of the BP upon exposure to a microbial solution have been measured. As such, these ROS species are undoubtedly produced as the BP materials decompose in the presence of microbial solution, such as that studied here. Both AFM and SEM investigation support these degradative process (see FIGS. 8 and 9). As such, it is proposed that the mechanism of anti-microbial activity is the production of ROS species, which is the driving factor behind the high degrees of cell lysis observed in these studies (c.f. FIGS. 3 to 5, and 7).

For the bacterial and fungal species investigated, this anti-microbial action renders most surface attached cells non-viable to a high degree of efficacy; however, some differences in activity are noted between species. The bacteria E. coli, P. aeruginosa, MRSA, S. typhimurium, and the fungi C. albicans, C. auris, C. neoformans (Sensitive), C. neoformans F^{R}, and C. neoformans A^{R} were all at least 90% non-viable on average, with some species showing higher degrees of susceptibility to the action of the nanomaterial. Interestingly, B. cereus cells were found to be only ˜80% non-viable, on average, following 2 hours of treatment, indicating that the cells were more resistant to the anti-microbial action of the BP flakes. The genus Bacillus are known to sporulate in response to environmental stress.⁸²⁻⁸⁴ The presence of these spores is clearly seen in both the SEM and CLSM images of B. cereus systems.

The bacterial spores produced during this biological survival mechanism are highly resistant, dormant structures, which enable prolonged survival of these organisms in adverse environments,^{85, 86} such as that produced by the presence of the BP flakes. In this light, it is not surprising that the B. cereus cells were less susceptible to the antibacterial action of the solvent-free BP flakes; however, the level of inactivation achieved, are still considered high for a bactericidal effect towards spore forming bacteria. This can be further enhanced by using thinner BP layers at slightly higher concentrations which will produce higher number of ROS and possibly overcome resistance.

Mammalian cells, specifically L929 mouse fibroblasts and BJ-5TA human fibroblasts, along with RBCs were found to be unharmed by the presence of BP under the same conditions as the microbial cells. The proliferation rate for the BJ-5TA human fibroblast cells was slower than the control in the presence of BP but cell growth still occurred. This is an important distinction, as it shows that the antimicrobial action of the BP nanoflakes is not toxic towards fibroblast cells in this form and concentrations; however, the material can induce high degrees of microbial cell lysis. The results of these studies are comparatively highlighted in Table 3. Notably, the few-layer BP investigated here is in a purer form than the previously reported for composite-based studies,^{60, 81} and at a significantly lower concentration, without compromising the antimicrobial efficacy. Importantly, previous studies have shown that bacteria are more sensitive to some anti-microbial agents, such as silver, compared to mammalian cells.⁸⁷ This means that there is a therapeutic window where an anti-microbial material is effective against pathogens while remaining innocuous towards mammalian cells. It is noteworthy that mammalian cells are known to respond to oxidative stress, such as that imposed by ROS, in a different manner than pathogenic, microorganisms.⁸⁸ The precise nature of these differences is still unclear, and requires further systematic, in-depth studies, but are at least partly attributable to improved ability to sequester ROS and to repair ROS-related cellular damage.

The general utility of BP as an anti-microbial surface additive was established via functionalizing a medical-grade titanium surface with mechanically exfoliated BP. Critically, the anti-microbial activity of the BP was retained at the titanium interface (see FIG. 7). This is unsurprising, as the anti-microbial activity of BP is derived from the ability of the materials to produce ROS during the natural degradation processes, which occur in solution. To this end, it is evident that mechanically exfoliated BP has utility beyond laboratory-based testing and will likely find real-world applications as a surface additive anti-microbial material.

The mechanically exfoliated BP investigated in this study has several advantages over other nanomaterial based anti-microbials reported within the literature (see Table 3), including: 1) the material displays high levels of anti-microbial efficacy towards both bacterial and fungal species, 2) the concentration required to elicit a high anti-microbial response is significantly lower than used for other nanomaterials, 3) the fabrication method is simple, and requires no other processing or stabilisers to elicit the anti-microbial effects, saving on cost of production. This means that it is readily scalable and represents a simple fabrication procedure, making it an accessible production method worldwide, and 4) any supportive surface could be potentially used with the material, as long as it could be preserved in an inert environment prior to use.

Importantly, the solvent free BP flakes are also known to readily degrade as a function of time when exposed to ambient conditions,^{47-49, 52, 55-57, 64}, meaning that the material is by nature biodegradable. The data obtained in this study highlights the broad-spectrum anti-microbial activity of solvent-free BP flakes, with eight medically relevant pathogenic bacterial and fungal species investigated, including drug-sensitive and drug resistant strains. This highlights the wide-reaching utility of native BP as a biocompatible anti-microbial additive. Together, these data indicate solvent-free BP flakes to be a highly effect anti-microbial, which has scope for use in scientific, medical, and industrial settings.

TABLE 3
Comparative anti-microbial activity of commonly investigated nanomaterials.

| | Sizes/ | | | Microbes Tested | | |
|---|---|---|---|---|---|---|
| Materials | Thickness | Concentration | Activated | Bacteria | | Fungi |
| Mechanically | Size: 500 | nanograms | No | E. coli, P. | | C. albicans, |
| Exfoliated | nm-5 μm | | | aeruginosa, | | C. auris |
| BP | Thickness: | | | MRSA, S. | | and |
| | 15-90 nm | | | typhimurium, | | sensitive, |
| | | | | and B. | | fluconazole- |
| | | | | cereus | | resistant, |
| | | | | | | and |
| | | | | | | Amphotericin |
| | | | | | | B-resistant C. |
| | | | | | | neoformans |
| NMP-BP | Size: 220 | 50 μg/mL | No | E. coli & | | No |
| with | nm | | | S. aureus | | |
| Ti-SA₄⁶⁰ | Thickness: | | | | | |
| | 5 nm | | | | | |
| DMPU- | Size: 0.1- | 160 μg/mL | Yes; NIR | E. coli & | | No |
| BP⁵⁹ | 4 μm. | | irradiation | S. aureus | | |
| | Thickness: | | at 808 | | | |
| | 2-15.4 nm | | nm. | | | |
| Ag and BP | AgNPs: 30 | 25-40 | Yes; NIR | MRSA | | No |
| nanosheets⁶¹ | nm | μg/mL | irradiation | | | |
| | BP: 220 nm | | at 808 | | | |
| | Thickness: | | nm. | | | |
| | 4 nm | | | | | |
| DCM-BP | Size: | 100 μg/ml | Yes; | E. coli & | | No |
| with | microns | | irradiation | S. aureus | | |
| PPMS⁷⁷ | Thickness: | | at 660 nm | | | |
| | 4.2-4.5 | | | | | |
| | nm | | | | | |
| | Size: 215.8 | | | | | |
| Millipore | nm | 50-100 | No | E. coli & B. | | No |
| water-BP | Thickness: | μg/mL. | | subtilis | | |
| | 1.6 nm | | | | | |
| Au-BP | Size: >100 | <10 μg/mL | No | E. coli | | No |
| Nanosheets⁸⁹ | nm | | | | | |
| | Thickness: | | | | | |
| | 2 nm | | | | | |
| BP-TiO₂⁹⁰ | NR | 25 μg/mL | Yes; UV- | E. coli & S. | | No |
| | | | vis | aureus | | |
| MoS₂ | Size: NR | ≤1 mg/mL | Yes, | E. coli & S. | | No |
| Composites^{52, 58} | Thickness: | | NIR⁵⁸ | aureus | | |
| | 2.2 nm | | | | | |
| Ag NPs⁹³ | Size: 4-24 | 50 μg/mL | No | E. coli | | Yes⁹⁴⁻⁹⁷ |
| | nm | | | | | |
| Au NPs¹⁰⁰ | Size: 10- | Widely | No | Controversial¹⁰⁰ | | Yes;^{101, 102} |
| | 200 nm | Variant | | | | controversial |
| ZnO NPs^{46, 106} | Size: 50- | 0.25 g/L | Yes, UV- | E. coli & S. | | Numerous |
| | 250 nm | | vis^{46, 106} | aureus | | Species¹⁰⁷⁻¹⁰⁹ |
| Graphene | Size: ~0.31 | 80 μg/mL | No | E. coli | | Yes;¹¹⁴ |
| oxide | μm Size: | | | | | enhanced |
| (pure & | ~2.75 μm | | | | | with NIR¹¹⁵ |
| reduced)⁵⁰ | | | | | | |
| Graphene | Size: 5-20 | 200 μg/mL | No | None for | | NR |
| oxide⁵³ | μm | | | pure | | |
| | Thickness: | | | graphene | | |
| | 1.2 nm | | | oxide | | |
| TiO₂ NPs¹¹⁸ | Size: 79 nm | 1200 μM | UV-visible | E. coli | | Yes¹¹⁹, |
| | | | Light | | | surface |
| | | | | | | additive^{120, 121} |
| Cu—TiO₂ | Size: 15-50 | 1 mg/mL | UV-vis | E. coli | | NR |
| NPs¹²⁴ | nm | | | | | |
| Au | Size: | Monolayer | NIR laser | S. aureus | | NR |
| nanostar¹²⁵ | 50-100 nm | of nanostar | | | | |
| | | on glass | | | | |
| Au | Size: ~100 | 0.2 mg/mL | NIR laser | P. | | NR |
| nanocross¹²⁶ | nm | | | aeruginosa | | |
| | | | Treatment Parameters | | | |
| | Mammalian Testing | | | Bacterial | Bactericidal | |
| | Cells | | Bactericidal | Treatment | Fungicidal | Treatment |
| Materials | Tested | Cytotoxicity | Activity | Times | Activity | time |
| Mechanically | L929 | No | See Table | 2 hours | See Table | 2 hours |
| Exfoliated | fibroblasts | | 2 | | 2 | |
| BP | | | | | | |
| NMP-BP | No | NR | 99.2% (E. | 3 hr | NR | NR |
| with | | | coli), | | | |
| Ti-SA₄⁶⁰ | | | 94.6% (S. | | | |
| | | | aureus). | | | |
| DMPU-BP⁵⁹ | N | NR | 99.2% | 3-10 min | NR | NR |
| Ag and BP | RBC, | No | 93%. | 5 min | NR | NR |
| nanosheets⁶¹ | 4T1, L929 | | | | | |
| | and HeLa | | | | | |
| DCM-BP | In Vivo | No | 99.3% & | 10 min | NR | NR |
| with | Mouse | | 99.2% (E. | | | |
| PPMS⁷⁷ | Models | | coli), 76.5% | | | |
| | | | & 69.5% | | | |
| | | | (S. aureus). | | | |
| Millipore | No | NR | 91.65% & | 6-12 hr | NR | NR |
| water-BP | | | 99.69% | | | |
| Au-BP | No | NR | 94.7% | 8 hr | NR | NR |
| Nanosheets⁸⁹ | | | | | | |
| BP-TiO₂⁹⁰ | No | NR | NR | 70 min | NR | NR |
| MoS₂ | Epithelial | Concentration | 100% | ≤6 hr | NR | NR |
| Composites^{52, 58} | cells & in | & system | | | | |
| | vivo | dependent^{91, 92} | | | | |
| | mouse | | | | | |
| | models^{65, 66} | | | | | |
| Ag NPs⁹³ | Numerous⁹⁸ | Yes; | 100% | 24 hr | Varying | |
| | | shape & | | | degrees.^{96, 99} | |
| | | concentration | | | | |
| | | dependent⁹⁸ | | | | |
| Au NPs¹⁰⁰ | in vitro¹⁰³ | Dose & | NB | N/A | MIC: 4- | |
| | & in | size | | | 48 pg/mL | |
| | vivo¹⁰⁴ | dependent¹⁰⁵ | | | | |
| ZnO NPs^{46, 106} | Yes¹¹⁰⁻¹¹² | Yes¹¹⁰⁻¹¹² | Conditional, | 2 hr | Yes^{108, 109, 113} | |
| | | | but >99% | 2 hr | | |
| Graphene | HUVECS,¹¹⁶ | Morphology, | Pure: 90% | | IC₅₀: | |
| oxide | RBC & | chemistry, sys | Reduced: | | 50-100 | |
| (pure & | Fibroblasts¹¹⁷ | dependent^{116, 117} | 80% | | μg/mL¹¹⁴ | |
| reduced)⁵⁰ | | | | | | |
| Graphene | NR | NR | 0% | N/A | NR | NR |
| oxide⁵³ | | | | | | |
| TiO₂ NPs¹¹⁸ | human | Yes, | 75% | N/A | Yes^{119, 123} | |
| | lung | concentration- | reduction | | | |
| | epithelial | & time- | | | | |
| | cells, | dependent | | | | |
| | A549 | manner.¹²² | | | | |
| | carcinom | | | | | |
| | a cells & | | | | | |
| | L-132 | | | | | |
| | normal | | | | | |
| | cells¹²² | | | | | |
| Cu—TiO₂ | NR | NR | 100% | N/A | NR | NR |
| NPs¹²⁴ | | | reduction | | | |
| Au | NR | NR | 99% | 30 min | NR | NR |
| nanostar¹²⁵ | | | | | | |
| Au | NR | NR | 99% | 5 min | NR | NR |
| nanocross¹²⁶ | | | | | | |

Abbreviations: BP: black phosphorus, NMP: N-methyl-2-pyrrolidone, DMPI: N,N′-dimethylpropyleneurea, PPMS: 4-pyridonemethylstyrene, NPs: nano-particles, NB: Not bactericidal, N/A: Not applicable, NR: Not reported. The summary termed “Mechanically Exfoliated BP” is that of this study. The studies highlighted in respect of AU NPs and Graphene oxide represent work that highlights the controversial nature of a class of anti-microbial nanomaterials.

Applications

Based on these findings, the inventors believe that solvent-free black phosphorus flakes could be applied to a wide range of substrates for use in an equally wide range of applications where such high anti-microbial activity would be beneficial.

For instance, the inventors believe that solvent-free BP flakes could be applied to the surface of a surgical implant or medical instrument using an appropriate means, for example an adhesive.

Preferably, the substrate is selected from the group consisting of: metal, an alloy, a polymer, a textile, a glass, a ceramic, or any combination thereof.

Typically, many surgical implants and medical instruments are manufactured from a medical grade metal or alloy. As already demonstrated above, solvent-free BP flakes can be applied as an anti-microbial coating to the surface of titanium; a metal frequently used for implants and medical instruments.

Similarly, the inventors believe that the surfaces of other metal substrates could be modified with an anti-microbial coating prepared from solvent-free BP flakes. Such metals may include, but are not limited to gold, silver, stainless steel, aluminium, copper, or any combination thereof.

In other embodiments, the substrate to be modified with an anti-microbial coating prepared from solvent-free BP flakes may include a bioscaffold, typically formed from a polymer or composite material.

Similarly, the substrate may also be a woven or non-woven textile such as those used in the manufacture of, for example, bandages, medical garments, bed linen and surgical drapes.

### CONCLUSIONS

Black phosphorus flakes produced in a solvent-free environment has been explored as a versatile anti-microbial surface coating against both anti-microbial sensitive and resistant bacterial and fungal cells. Importantly, nanogram concentrations of the material were found to be highly anti-microbial towards a broad-spectrum of bacteria and fungi, including E. coli, P. aeruginosa, MRSA, S. typhimurium, and B. cereus, as well as the fungal strains, C. albicans, C. auris and sensitive, fluconazole-resistant, and Amphotericin B-resistant C. neoformans cells.

Further, the solvent-free BP material can be easily deposited onto industrially and medically important substrate surfaces, such as medical grade titanium, via a simple mechanical exfoliation procedure, to impart extremely effective anti-microbial character within 2 hours of microbial exposure.

The study demonstrates BP as a next-generation anti-microbial platform for the treatment of bio-interfacial infections, such as that of wound dressings, as well as for the on-demand sterilisation of applicable surfaces, such as medical devices. More broadly, this study provides a facile methodology for the anti-microbial functionalization of a substrate surface via the deposition of BP-based nanomaterials.

Methods and Materials

Phosphorus Synthesis

BP flakes were produced via mechanical exfoliation from a bulk BP crystal (SmartElements). For microbial testing, the BP flakes were exfoliated directly onto glass bottomed petri-dishes (FluoroDish Cell Culture dishes, Part Number: FD35-100, World Precision Instruments, Sarasota, Fla., U.S.A.).

For SEM imaging, the BP flakes were exfoliated onto plasma-cleaned 300 nm SiO₂/Si substrates prior to incubation and SEM preparation. BP surfaces undergo photo-oxidisation in ambient conditions, so the samples were prepared and stored in a nitrogen, enclosed (dark, UV protected)) atmosphere prior to use. BP flakes of uniform thicknesses of 25 to 30 nm were identified via optical contrast microscopy. In the field of electronics, few-layer BP is notoriously known to degrade under ambient conditions.⁷⁴ As such, the exfoliation process was carried out in UV deficient, dark conditions and the samples were stored in inert nitrogen (N₂) atmosphere to preserve the integrity of the material prior to use.^{67, 68}

Bacterial Strains, Growth Conditions, and Sample Preparation

All bacterial strains were obtained from the American Type Culture Collection. Specifically, the stains Escherichia coli DH52, Pseudomonas aeruginosa ATCC27853, Methicillin-resistant Staphylococcus aureus ATCC700699, Salmonella typhimurium ATCC13311, and Bacillus cereus ATCC11778 were investigated in the study. This library of species were chosen as medically relevant pathogenic species which contains representatives of both Gram-negative and Gram-positive bacterium.¹²⁷

Further, they represent the two main morphologies among bacteria: rod and cocci cells, respectively, for comparison, as well as sporulation in the case of B. cereus. For each experiment, bacteria cultures were grown on Luria-Bertani (LB) agar overnight at 37° C. Bacterial cells were collected from the culture via an inoculation loop and suspended in nutrient broth. These planktonic cell suspensions were grown overnight at 37° C. in 5 mL of Luria-Bertani (LB) broth (B.D., U.S.A.) from loop. The density of the bacterial suspensions was then adjusted to OD600=0.1, after collection on at the logarithmic stage of cell growth.

To obtain a mature biofilm, the planktonic cell suspensions were then added into individual glass-bottom Petri dishes (FluoroDish Cell Culture dishes, Part Number: FD35-100, World Precision Instruments, Sarasota, Fla., U.S.A.) which were either bare or coated in few-layer BP, as indicated. Petri dishes were 35 mm in diameter with a 23 mm well, were comprised of plastic walls with a glass-bottom, and importantly were not pre-coated with any materials.

Fungal Strains, Growth Conditions, and Sample Preparation

Fluconazole- and amphotericin B-resistant Cryptococcus neoformans strains C. neoformans strains were obtained from a fluconazole and amphotericin B resistant isolates derived from strain KN99a which was originally produced by Nielsen et al. (K. Nielsen, G. M. Cox, P. Wang, D. L. Toffaletti, J. R. Perfect and J. Heitman, Infection and immunity, 2003, 71, 4831-4841).

The fungi Candida albicans and Candida auris clinical isolates were obtained from South Australia Pathology Laboratory. Fungal cultures were cultured on yeast extract-peptone-dextrose (YPD) plates for 2 days at 30° C. Fungal suspensions were made in YPD liquid medium with the adjusted OD₆₀₀=0.1. The incubation procedures on BP surfaces were carried out in a manner similar to that used in respect of the bacterial studies.

SEM Characterization

Scanning electron micrographs were obtained using a field-emission scanning electron microscope (FE-SEM). A FEI Verios Scanning Electron Microscopy (VP, Oberkochen, BW, Germany) at 5 kV was used to image the systems using methods previously described.^{26, 49, 128}

Prior to SEM imaging, all samples were chemically fixed, using 3% glutaraldehyde and 3% formaldehyde in sodium cacodylate buffer pH 7.4 (ProSciTec, QLD, Australia), following with dehydration with series of ethanol concentrations (30%, 50%, 70%, 90%, 100%, 100%). Dehydrated samples were further coated with a thin film of gold prior to imaging.

STEM Characterization

TEM images were obtained with a JEOL 2100F microscope (JOEL, Musashino, Akishima, Tokyo, Japan) equipped with a Gatan Orius SC1000 CCD camera and operated at an acceleration voltage of 80 keV. Images were processed and analysed using Digital Micrograph 2.31.

EELS Characterization

EELS data was collected with a nominal spot size of 1.5 nm and spectrometer entrance aperture of 5 mm with a dispersion of 0.3 eV/ch.

AFM Characterization

AFM images were obtained using both a Cypher ES AFM (Oxford Instrument, Asylum Research, Santa Barbara, Calif., USA) and a JPK nanowizard 4 (JPK BioAFM Business, Am Studio 2D, 12489 Berlin, Germany). All images were obtained under ambient conditions. AC240 cantilevers (Oxford Instrument, Asylum Research, Santa Barbara, Calif., USA, nominal spring constant k_{c}=2 N/m) were used for all measurements. When operated in AC mode imaging forces were minimized via a setpoint ratio (Imaging Amplitude (A)/free amplitude (AO)) of >0.7. The JPK instrument was operated in QI mode. All cantilevers were tuned prior to use using the thermal spectrum method in combination with inverse level sensitivity (InVOLs) as measured by force spectroscopy.

Confocal Imaging and Bacterial Cell Viability Analysis

A combination of confocal laser scanning microscopes (CLSM)—A fluoview FV1200 inverted microscope, Olympus, Tokyo, Japan, and a ZEISS LSM 880 Airyscan upright microscope, Oberkochen, Germany—were used to evaluate the proportions of live and dead cells in each bacteria prior to and following exposure to the polymers within a glass-bottom petri dish (FluoroDish Cell Culture dishes, Part Number: FD35-100, World Precision Instruments, Sarasota, Fla., U.S.A.). Cells were dyed using a LIVE/DEAD® BacLight™ Bacterial Viability Kit (including SYTO® 9 and propidium iodide) (Molecular Probes™, Invitrogen, Grand Island, N.Y., USA). Specifically, SYTO® 9 permeated both intact and damaged cell membranes, binding to nucleic acids and fluorescing green when excited by a 485 nm wavelength laser. Propidium iodide (PI) dominantly enters cells that have undergone membrane damage, which are considered to be non-viable, and binds with higher affinity to nucleic acids than SYTO® 9.

Bacterial suspensions were stained according to the manufacturer's protocol.¹²⁹ Importantly, discrepancies in viability assessment were avoided by ensuring that no green (485 nm) and red (543 nm) fluorescence overlap was observed during image assessment. Furthermore, photobleaching of the SYTO® 9 dye was avoided by limiting each surface location to a single confocal scan. The live and dead cell ratio was quantified using Cell-C (https://sites.google.com/site/cellcsoftware/) providing a meaningful assessment of the antibacterial activity of the surface.^{130, 131}

Raman Spectroscopy

The Raman spectroscopy characterizations were performed using a Horiba LabRAM HR Evolution Micro-Raman system equipped with a 532 nm laser source (100× objective).

ROS detection: The generation of oxidative species via few-layer BP decomposition under dark condition was determined using a series of dyes. The three oxidative species of interest were OH. radical, O₂.— radical and ¹O₂, and were screened for using horseradish peroxidase (HRP) enzyme (Sigma), xanthine oxidase (Sigma) and methylene blue (MB) (Sigma) respectively. Initially, few-layer BP was exfoliated onto 1 cm² piece of Si wafer and kept in the dark conditions prior to testing. The HRP solution was prepared by diluting 5 mg of the HRP was diluted into 50 mL of milliQ water. The xanthine oxidase solution was prepared by diluting 5 μM of the oxidase in 50 mL of milliQ water. Methyl blue solution was prepared by diluting 100 mg of the MB into 50 mL of milliQ water, then diluting by a factor of 10. The BP treated Si was covered with 3 mL of the prepared solutions, covered and incubated for 2 hours at room temperature. Bare Si wafter was used as the control. Following the incubation, 1 mL of each solution was run through the UV-visible spectrometer (CARY3500 UV-vis spectrophotometer) over a wavelength range of 200-800 nm. The absorbance peak associated with OH. was 350-450 nm, O₂.— was 250-350 nm and ¹O₂ was 500-700 nm.⁷³

Computational Methods: Quantum chemical calculations involving the few-layer BP surface were performed using density functional theory as implemented in the Vienna ab initio Simulation Package (VASP5.4.4).^{131, 139}. The generalized-gradient (GGA) approximation was employed with the Perdew-Burke-Ernzerhof (PBE) exchange-correlation functional¹⁴⁰ and projector augmented wave (PAW) method¹⁴¹ to define the ion-electron interaction. An energy cutoff of 466 eV was applied, with a k-point mesh of 5×5×1 for geometry optimizations and 3×3×1 for ab initio molecular dynamics (AIMD) to sample the Brillouin zone. The van der Waals forces were accounted for by the Grimme DFT-D3 approach.¹⁴² In all cases the lattice remained fixed to the previously optimized 2×3 supercell of a single layer of BP, with lattice parameters of a=9.895 Å, b=9.245 Å, and c=20 Å, which included a vacuum region of ˜15 Å to minimize interlayer interactions.¹⁴³ This system was found to have a bandgap of 0.89 eV, consistent with previous studies. The single-defect BP layer was created by removing a single P atom from the top of the pristine BP. Partially oxidized surfaces were created by changing surface P atoms to O, and optimizing the geometry. For geometry optimization calculations, all atomic positions were relaxed until the total energy of the system was converged to 10-4 eV and the Hellman-Feynman force on each relaxed atom was less than 0.01 eV/A. For the AIMD simulations, two molecules of water and two molecules of O₂ were added to each system and all atoms were allowed to relax during the simulation. The simulations were performed for 10 μs at 300 K using a time step of 1 fs.

Mammalian Cell Testing (L929 Mouse Fibroblasts)

The L929 fibroblasts were used to test in vitro cytotoxicity of BP flakes. Cell culture media was DMEM solution supplemented with 10% FBS, and 1% penicillin/streptomycin. The cells were grown to 80% confluence before being collected for experiments.

Prior to the cell experiments, BP flakes were exfoliated onto the well of a sterile tissue culture treated 24-well polystyrene plate. L929 cells were seeded at a concentration of 10,000 cells/cm² and were incubated at 37° C., 5% CO₂ for 48 hours. A sterile well plate with no BP flakes were used as a control. L929 cell viability was quantified by using an MTS assays. For this, 100 μL of CellTiter 96@ AQueous One Solution Proliferation Assay (Promega) was added to each well of the 24-well plates. The plates were incubated at 37° C., in the dark for three hours then analysed using a microplate reader (Spectramax Paradigm) at 490 nm absorbance. The absorbance was normalized to that measured from the control cells.

The cells were also visualized using a confocal laser scanning microscope. The cells were first rinsed with PBS and fixed with formalin 4% solution for 15 mins. They were rinsed twice with PBS to remove excess formalin. The cells were permeabilized and blocked with 0.1% Triton X-100 and 1% bovine serum albumin (BSA), respectively, and were washed three times with PBS. Rhodamine Phalloidin and DAPI (ThermoFisher) were used to stain actin filaments and nucleus, respectively. The samples were washed with PBS and stored with 1 mL of PBS at 4° C. for fluorescent microscopy imaging (Zeiss). DAPI staining was identified as blue when assessed via CLSM and Rhodamine Phalloidin was identified as red when assessed via CLSM.

Mammalian Cell Testing (BJ-5TA human fibroblasts): The BJ-5TA fibroblasts (ATCC-CRL 4001) were used to further test the in vitro cytotoxicity of the BP flakes. Cell culture used was DMEM (ThermoFisher) in a 4:1 ratio with Medium 199 (ThermoFisher), supplemented with hygromycin and 10% foetal bovine serum. Prior to the cell experiment, BP flakes were exfoliated onto half the wells of a sterile tissue cell culture 96-well polystyrene plate, with the remaining bare wells used as controls. BJ-5TA cells were seeded at a concentration of 10,000 cells cm⁻² and were incubated at 37° C., 5% CO₂ for 48 hours. BJ-5TA cell viability was assessed after 48 hours of exposure using 2 μM Calcein-AM (Cayman Chemicals) and 4 μM Ethidium homodimer (Sigma-Aldrich) added into each well and incubated for 30 min in a 37° C., 5% CO₂ incubator. The Calcein-AM is a cell-permeable dye that is hydrolysed by intracellular esterases into green-fluorescent calcein under 494 nm wavelength laser. Ethidium homodimer is a cell-impermeable dye that binds to cellular DNA and fluoresces reddish-orange under 493 nm wavelength laser. The cells were visualized using a high throughput fluorescence microscope (Operetta CLS, PerkinElmer) and were counted using the Harmony software (Operetta CLS, PerkinElmer).

Hemolysis: Fresh blood was collected from a donor of this study and analyzed following the protocol outlined by Li et. al.⁷⁹ Briefly, the RBCs were separated by centrifuging at 1500 rpm for 15 min then the RBCs were washed with sterile PBS three times. The RBCs were then resuspended in 6 mL of PBS and 1.5 mL of the RBC suspension was exposed to either bare glass cover-slips or BP deposited glass cover-slips (1 cm²). Negative controls were obtained by adding 2 mL of ethanol to the RBC suspensions. After incubating for 2 hours at 37° C., 1 mL of the RBC solution was removed and diluted with 2 mL sterile PBS. The absorbance was measured at a wavelength of 576 nm using a CARY3500 UV-vis spectrophotometer.

Medically Relevant Substrates.

ASTM commercially pure grade 2 titanium rods with nominal diameter of 10 mm were used to manufacture Ti substrates. Firstly, Ti rods were cut into individual discs with an approximate thickness of 5 mm using a Secotom 50 cutting machine (Struers, GmbH, Willich, Germany). Ti discs were then polished with silicon carbide grinding papers with a grit size of P1200 until medical grade smoothness was reached. Each Ti discs was then sterilized via ultrasonication successive washes of MilliQ water and ethanol and then exposed to a UV light source for 30 minutes. Discs were then allowed to dry in a Biosafety Cabinet Class II for 12 hours prior to use. Sylgard™ 184 silicone encapsulant kit (Sigma-Aldrich Australia) was used to cure the PDMS, using a 10:1 ratio of Silicon to a curing agent, and was incubated at 37 C for 24 hours. A 1 cm² piece was then cut and sterilized via soaking in ethanol and allowed to dry in a Biosafety Cabinet Class II for 30 mins before use.

Commercial grade PET (local source) was obtained, a 1 cm² area was cut out and sterilized via soaking in ethanol and allowed to dry in a Biosafety Cabinet Class II for 30 mins prior to use. Following sterilization, the surfaces were exposed to 1 mL of microbial solution (prepared using the same procedure above) and incubated for 2 hours under dark conditions. After the incubation period, the surfaces were gently rinsed with sterile PBS and prepared for CLSM images (mentioned above). A commercial-grade bandage (Coverplast standard, BSN medical) was obtained and remained sealed before experimentation. The top layer of the bandage pad was used as the deposition site for the BP flakes. Initially, the 10 uL of the microbial solution was spread onto the corresponding agar plate (LB agar for MRSA and YPD for C. neoformans(F^{R})) and air-dried for 30 seconds. After the excess liquid dried, the bandage was placed onto the agar and incubated overnight (˜16 hours) at 25° C. The bandage was then removed and placed into a sterile glass-bottom petri dish and prepared for CLSM imaged following the outlined procedure above.

### REFERENCES

1. Costerton, J. W., Stewart, P. S. & Greenberg, E. P. Bacterial Biofilms: A Common Cause of Persistent Infections. Science 284, 1318-1322 (1999).
2. Hall-Stoodley, L., Costerton, J. W. & Stoodley, P. Bacterial biofilms: from the natural environment to infectious diseases. Nature reviews microbiology 2, 95-108 (2004).
3. Bridier, A., Briandet, R., Thomas, V. & Dubois-Brissonnet, F. Resistance of bacterial biofilms to disinfectants: a review. Biofouling 27, 1017-1032 (2011).
4. Campoccia, D., Montanaro, L. & Arciola, C. R. The Significance of Infection Related to Orthopedic Devices and Issues of Antibiotic Resistance. Biomaterials 27, 2331-2339 (2006).
5. Elbourne, A., Crawford, R. J. & Ivanova, E. P. Nano-structured antimicrobial surfaces: From nature to synthetic analogues. Journal of Colloid and Interface Science 508, 603-616 (2017).
6. Boucher, H. W. et al. Bad Bugs, No Drugs: No ESKAPE! An Update from the Infectious Diseases Society of America. Clinical Infectious Diseases 48, 1-12 (2009).
7. Spellberg, B. et al. The Epidemic of Antibiotic-Resistant Infections: A Call to Action for the Medical Community from the Infectious Diseases Society of America. Clinical Infectious Diseases 46, 155-164 (2008).
8. Levy, S. B. & Marshall, B. Antibacterial resistance worldwide: causes, challenges and responses. Nat Med (2004).
9. Control, C.f.D. & Prevention Antibiotic resistance threats in the United States, 2013. (Centres for Disease Control and Prevention, US Department of Health and Human Services, 2013).
10. Neu, H. C. The crisis in antibiotic resistance. Science 257, 1064-1074 (1992).
11. Bush, K. et al. Tackling antibiotic resistance. Nature Reviews Microbiology 9, 894-896 (2011).
12. Health, U. D.o. & Services, H. Antibiotic resistance threats in the United States, 2013. Atlanta: CDC (2013).
13. Gupta, N., Limbago, B. M., Patel, J. B. & Kallen, A. J. Carbapenem-Resistant Enterobacteriaceae: Epidemiology and Prevention. Clinical Infectious Diseases 53, 60-67 (2011).
14. Jacob, J. T. et al. Vital signs: Carbapenem-resistant enterobacteriaceae. Morbidity and Mortality Weekly Report 62, 165-169 (2013).
15. Enright, M. C. et al. The evolutionary history of methicillin-resistant Staphylococcus aureus (MRSA). Proceedings of the National Academy of Sciences 99, 7687-7692 (2002).
16. Klevens, R. M. et al. Invasive methicillin-resistant Staphylococcus aureus infections in the United States. Jama 298, 1763-1771 (2007).
17. Rodvold, K. A. & McConeghy, K. W. Methicillin-Resistant Staphylococcus aureus Therapy: Past, Present, and Future. Clinical Infectious Diseases 58, S20-S27 (2014).
18. Humphreys, G. & Fleck, F. United Nations meeting on antimicrobial resistance. World Health Organization. Bulletin of the World Health Organization 94, 638 (2016).
19. O'Neill, J. Tackling drug-resistant infections globally: Final report and recommendations. 2016. HM Government and Welcome Trust: UK (2018).
20. Boyce, K., Morrissey, O., Idnurm, A. & Macreadie, I. Insights into the global emergence of antifungal drug resistance. Microbiology Australia.
21. Brown, G. D. et al. Hidden killers: human fungal infections. Science translational medicine 4, 165rv113-165rv113 (2012).
22. Slavin, M. et al. Burden of hospitalization of patients with Candida and Aspergillus infections in Australia. International journal of infectious diseases 8, 111-120 (2004).
23. Ananda-Rajah, M. R. et al. Attributable hospital cost and antifungal treatment of invasive fungal diseases in high-risk hematology patients: an economic modeling approach. Antimicrobial agents and chemotherapy 55, 1953-1960 (2011).
24. Elbourne, A. et al. Bacterial-nanostructure interactions: the role of cell elasticity and adhesion forces. Journal of Colloid and Interface Science 546, 192-210 (2019).
25. Elbourne, A. et al. in Methods in Microbiology (Academic Press, 2019).
26. Elbourne, A. et al. Multi-directional electrodeposited gold nanospikes for antibacterial surface applications. Nanoscale Advances (2018).
27. Murata, H., Koepsel, R. R., Matyjaszewski, K. & Russell, A. J. Permanent, non-leaching antibacterial surfaces-2: How high density cationic surfaces kill bacterial cells. Biomaterials 28, 4870-4879 (2007).
28. Willcox, M., Hume, E., Aliwarga, Y., Kumar, N. & Cole, N. A novel cationic-peptide coating for the prevention of microbial colonization on contact lenses. Journal of applied microbiology 105, 1817-1825 (2008).
29. KAlicke, T. et al. Effect on infection resistance of a local antiseptic and antibiotic coating on osteosynthesis implants: An in vitro and in vivo study. Journal of Orthopaedic Research 24, 1622-1640 (2006).
30. Zhao, L., Chu, P. K., Zhang, Y. & Wu, Z. Antibacterial coatings on titanium implants. Journal of Biomedical Materials Research Part B: Applied Biomaterials 91B, 470-480 (2009).
31. Nederberg, F. et al. Biodegradable nanostructures with selective lysis of microbial membranes. Nat Chem 3, 409-414 (2011).
32. Banerjee, I., Pangule, R. C. & Kane, R. S. Antifouling Coatings: Recent Developments in the Design of Surfaces That Prevent Fouling by Proteins, Bacteria, and Marine Organisms. Advanced Materials 23, 690-718 (2011).
33. Tiller, J. C., Liao, C.-J., Lewis, K. & Klibanov, A. M. Designing surfaces that kill bacteria on contact. Proceedings of the National Academy of Sciences 98, 5981-5985 (2001).
34. Nguyen, L. T., Haney, E. F. & Vogel, H. J. The expanding scope of antimicrobial peptide structures and their modes of action. Trends in Biotechnology 29, 464-472 (2011).
35. Gao, G. et al. The biocompatibility and biofilm resistance of implant coatings based on hydrophilic polymer brushes conjugated with antimicrobial peptides. Biomaterials 32, 3899-3909 (2011).
36. Norowski, P. A. & Bumgardner, J. D. Biomaterial and antibiotic strategies for peri-implantitis: A review. Journal of Biomedical Materials Research Part B: Applied Biomaterials 88B, 530-543 (2009).
37. Oosterbos, C. et al. Osseointegration of hydroxyapatite-coated and noncoated Ti6Al4V implants in the presence of local infection: a comparative histomorphometrical study in rabbits. Journal of Biomedical Materials Research Part A 60, 339-347 (2002).
38. Stigter, M., De Groot, K. & Layrolle, P. Incorporation of tobramycin into biomimetic hydroxyapatite coating on titanium. Biomaterials 23, 4143-4153 (2002).
39. Stigter, M., Bezemer, J., De Groot, K. & Layrolle, P. Incorporation of different antibiotics into carbonated hydroxyapatite coatings on titanium implants, release and antibiotic efficacy. Journal of controlled release 99, 127-137 (2004).
40. Knetsch, M. L. W. & Koole, L. H. New Strategies in the Development of Antimicrobial Coatings: The Example of Increasing Usage of Silver and Silver Nanoparticles. Polymers 3, 340 (2011).
41. Johnston, H. J. et al. A review of the in vivo and in vitro toxicity of silver and gold particulates: particle attributes and biological mechanisms responsible for the observed toxicity. Critical reviews in toxicology 40, 328-346 (2010).
42. Kuo, W.-S., Chang, C.-N., Chang, Y.-T. & Yeh, C.-S. Antimicrobial gold nanorods with dual-modality photodynamic inactivation and hyperthermia. Chemical Communications, 4853-4855 (2009).
43. Anghel, I. et al. Biohybrid Nanostructured Iron Oxide Nanoparticles and Satureja hortensis to Prevent Fungal Biofilm Development. International Journal of Molecular Sciences 14, 18110 (2013).
44. Pal, S., Tak, Y. K. & Song, J. M. Does the antibacterial activity of silver nanoparticles depend on the shape of the nanoparticle? A study of the gram-negative bacterium Escherichia coli. Applied and environmental microbiology 73, 1712-1720 (2007).
45. Govindaraju, S., Ramasamy, M., Baskaran, R., Ahn, S. J. & Yun, K. Ultraviolet light and laser irradiation enhances the antibacterial activity of glucosamine-functionalized gold nanoparticles. International journal of nanomedicine 10, 67 (2015).
46. Ozkan, E., Allan, E. & Parkin, I. P. White-Light-Activated Antibacterial Surfaces Generated by Synergy between Zinc Oxide Nanoparticles and Crystal Violet. ACS Omega 3, 3190-3199 (2018).
47. Zhao, Y. et al. Near-Infrared Light-Activated Thermosensitive Liposomes as Efficient Agents for Photothermal and Antibiotic Synergistic Therapy of Bacterial Biofilm. ACS applied materials & interfaces 10, 14426-14437 (2018).
48. Dai, X. et al. All-in-one NIR-activated nanoplatforms for enhanced bacterial biofilm eradication. Nanoscale (2018).
49. Rajapaksha, P. et al. Antibacterial Properties of Graphene Oxide-Copper Oxide Nanoparticle Nanocomposites. ACS Applied Bio Materials (2019).
50. Liu, S. et al. Antibacterial activity of graphite, graphite oxide, graphene oxide, and reduced graphene oxide: membrane and oxidative stress. ACS nano 5, 6971-6980 (2011).
51. Yang, Z. et al. Long-term antibacterial stable reduced graphene oxide nanocomposites loaded with cuprous oxide nanoparticles. Journal of Colloid and Interface Science 533, 13-23 (2019).
52. Kim, T. I. et al. Antibacterial Activities of Graphene Oxide-Molybdenum Disulfide Nanocomposite Films. ACS Applied Materials & Interfaces 9, 7908-7917 (2017).
53. Barbolina, I. et al. Purity of graphene oxide determines its antibacterial activity. 2D Materials 3, 025025 (2016).
54. Hui, L. et al. Availability of the basal planes of graphene oxide determines whether it is antibacterial. ACS applied materials & interfaces 6, 13183-13190 (2014).
55. Tang, J. et al. Graphene oxide-silver nanocomposite as a highly effective antibacterial agent with species-specific mechanisms. ACS applied materials & interfaces 5, 3867-3874 (2013).
56. Liu, S. et al. Lateral Dimension-Dependent Antibacterial Activity of Graphene Oxide Sheets. Langmuir 28, 12364-12372 (2012).
57. Xu, W.-P. et al. Facile synthesis of silver@ graphene oxide nanocomposites and their enhanced antibacterial properties. Journal of Materials Chemistry 21, 4593-4597 (2011).
58. Zhang, W. et al. Versatile molybdenum disulfide based antibacterial composites for in vitro enhanced sterilization and in vivo focal infection therapy. Nanoscale 8, 11642-11648 (2016).
59. Sun, Z. et al. New solvent-stabilized few-layer black phosphorus for antibacterial applications. Nanoscale 10, 12543-12553 (2018).
60. Li, Z. et al. Synergistic Antibacterial Activity of Black Phosphorus Nanosheets Modified with Titanium Aminobenzenesulfanato Complexes. ACS Applied Nano Materials 2, 1202-1209 (2019).
61. Ouyang, J. et al. A black phosphorus based synergistic antibacterial platform against drug resistant bacteria. Journal of Materials Chemistry B 6, 6302-6310 (2018).
62. Choi, J. R. et al. Black phosphorus and its biomedical applications. Theranostics 8, 1005 (2018).
63. Luo, M., Fan, T., Zhou, Y., Zhang, H. & Mei, L. 2D Black Phosphorus-Based Biomedical Applications. Advanced Functional Materials 29, 1808306 (2019).
64. Anju, S., Ashtami, J. & Mohanan, P. Black phosphorus, a prospective graphene substitute for biomedical applications. Materials Science and Engineering: C (2019).
65. Naskar, A. & Kim, K.-s. Black phosphorus nanomaterials as multi-potent and emerging platforms against bacterial infections. Microbial Pathogenesis 137, 103800 (2019).
66. Walia, S. et al. Defining the role of humidity in the ambient degradation of few-layer black phosphorus. 2D Materials 4, 015025 (2016).
67. Ahmed, T. et al. Optically Stimulated Artificial Synapse Based on Layered Black Phosphorus. Small, 1900966 (2019).
68. Ahmed, T. et al. Multifunctional Optoelectronics via Harnessing Defects in Layered Black Phosphorus. Advanced Functional Materials, 1901991 (2019).
69. tapiriska, A., Taube, A., Judek, J. & Zdrojek, M. Temperature evolution of phonon properties in few-layer black phosphorus. The Journal of Physical Chemistry C 120, 5265-5270 (2016).
70. Kuriakose, S. et al. Effects of plasma-treatment on the electrical and optoelectronic properties of layered black phosphorus. Applied Materials Today 12, 244-249 (2018).
71. Lu, W. et al. Plasma-assisted fabrication of monolayer phosphorene and its Raman characterization. Nano Research 7, 853-859 (2014).
72. Elbourne, A. et al. Multi-directional electrodeposited gold nanospikes for antibacterial surface applications. Nanoscale Advances 1, 203-212 (2019).
73. Walia, S. et al. Ambient protection of few-layer black phosphorus via sequestration of reactive oxygen species. Advanced Materials 29, 1700152 (2017).
74. Kuriakose, S. et al. Black phosphorus: ambient degradation and strategies for protection. 2D Materials 5, 032001 (2018).
75. Ahmed, T. et al. Degradation of black phosphorus is contingent on UV-blue light exposure. npj 2D Materials and Applications 1, 18 (2017).
76. Tripathy, A., Sen, P., Su, B. & Briscoe, W. H. Natural and bioinspired nanostructured bactericidal surfaces. Advances in colloid and interface science 248, 85-104 (2017).
77. Tan, L. et al. In Situ Disinfection through Photoinspired Radical Oxygen Species Storage and Thermal-Triggered Release from Black Phosphorous with Strengthened Chemical Stability. Small 14, 1703197 (2018).
78. Xiong, Z. et al. Bacterial toxicity of exfoliated black phosphorus nanosheets. Ecotoxicology and environmental safety 161, 507-514 (2018).
79. Elbadawi, C. et al. Encapsulation-free stabilization of few-layer black phosphorus. ACS applied materials & interfaces 10, 24327-24331 (2018).
80. Wood, J. D. et al. Effective passivation of exfoliated black phosphorus transistors against ambient degradation. Nano letters 14, 6964-6970 (2014).
81. Alsaffar, F. et al. Raman Sensitive Degradation and Etching Dynamics of Exfoliated Black Phosphorus. Scientific Reports 7, 44540 (2017).
82. Andersson, A., Ronner, U. & Granum, P. E. What problems does the food industry have with the spore-forming pathogens Bacillus cereus and Clostridium perfringens? International journal of food microbiology 28, 145-155 (1995).
83. de Vries, Y. P., Hornstra, L. M., de Vos, W. M. & Abee, T. Growth and Sporulation of Bacillus cereus ATCC 14579 under Defined Conditions: Temporal Expression of Genes for Key Sigma Factors. Applied and Environmental Microbiology 70, 2514 (2004).
84. Leggett, M. J., McDonnell, G., Denyer, S. P., Setlow, P. & Maillard, J.-Y. Bacterial spore structures and their protective role in biocide resistance. Journal of Applied Microbiology 113, 485-498 (2012).
85. Errington, J. Bacillus subtilis sporulation: regulation of gene expression and control of morphogenesis. Microbiological reviews 57, 1-33 (1993).
86. Boylan, S. A., Redfield, A. R., Brody, M. S. & Price, C. W. Stress-induced activation of the sigma B transcription factor of Bacillus subtilis. Journal of bacteriology 175, 7931-7937 (1993).
87. Tran, N. et al. Silver doped titanium oxide-PDMS hybrid coating inhibits Staphylococcus aureus and Staphylococcus epidermidis growth on PEEK. Materials Science and Engineering: C 49, 201-209 (2015).
88. Sigler, K., Chaloupka, J., Brozmanovi, J., Stadler, N. & Höfer, M. Oxidative stress in microorganisms—I. Folia Microbiologica 44, 587-624 (1999).
89. Wu, Q., Liang, M., Zhang, S., Liu, X. & Wang, F. Development of functional black phosphorus nanosheets with remarkable catalytic and antibacterial performance. Nanoscale 10, 10428-10435 (2018).
90. Uk Lee, H. et al. Stable semiconductor black phosphorus (BP)@titanium dioxide (TiO2) hybrid photocatalysts. Scientific Reports 5, 8691 (2015).
91. Teo, W. Z., Chng, E. L. K., Sofer, Z. & Pumera, M. Cytotoxicity of Exfoliated Transition-Metal Dichalcogenides (MoS2, WS2, and WSe2) is Lower Than That of Graphene and its Analogues. Chemistry—A European Journal 20, 9627-9632 (2014).
92. Wang, X. et al. Differences in the Toxicological Potential of 2D versus Aggregated Molybdenum Disulfide in the Lung. Small 11, 5079-5087 (2015).
93. Sondi, I. & Salopek-Sondi, B. Silver nanoparticles as antimicrobial agent: a case study on E. coli as a model for Gram-negative bacteria. Journal of colloid and interface science 275, 177-182 (2004).
94. Kim, S. W. et al. Antifungal Effects of Silver Nanoparticles (AgNPs) against Various Plant Pathogenic Fungi. Mycobiology 40, 53-58 (2012).
95. Mallmann, E. J. J. et al. Antifungal activity of silver nanoparticles obtained by green synthesis. Rev Inst Med Trop Sao Paulo 57, 165-167 (2015).
96. Panáček, A. et al. Antifungal activity of silver nanoparticles against Candida spp. Biomaterials 30, 6333-6340 (2009).
97. Artunduaga Bonilla, J. J., Paredes Guerrero, D. J., Sanchez Suirez, C. I., Ortiz López, C. C. & Torres Siez, R. G. In vitro antifungal activity of silver nanoparticles against fluconazole-resistant Candida species. World Journal of Microbiology and Biotechnology 31, 1801-1809 (2015).
98. Zhang, T., Wang, L., Chen, Q. & Chen, C. Cytotoxic potential of silver nanoparticles. Yonsei Med J 55, 283-291 (2014).
99. Xia, Z.-K. et al. The antifungal effect of silver nanoparticles on Trichosporon asahii. Journal of Microbiology, Immunology and Infection 49, 182-188 (2016).
100. Zhang, Y., Shareena Dasari, T. P., Deng, H. & Yu, H. Antimicrobial activity of gold nanoparticles and ionic gold. Journal of Environmental Science and Health, Part C 33, 286-327 (2015).
101. Ahmad, T. et al. Antifungal activity of gold nanoparticles prepared by solvothermal method. Materials Research Bulletin 48, 12-20 (2013).
102. Peña-González, C. E. et al. Antibacterial and antifungal properties of dendronized silver and gold nanoparticles with cationic carbosilane dendrons. International Journal of Pharmaceutics 528, 55-61 (2017).
103. Falagan-Lotsch, P., Grzincic, E. M. & Murphy, C. J. One low-dose exposure of gold nanoparticles induces long-term changes in human cells. Proceedings of the National Academy of Sciences 113, 13318-13323 (2016).
104. Wang, J.-Y. et al. Effects of surface charges of gold nanoclusters on long-term in vivo biodistribution, toxicity, and cancer radiation therapy. International journal of nanomedicine 11, 3475 (2016).
105. Pan, Y. et al. Size-dependent cytotoxicity of gold nanoparticles. Small 3, 1941-1949 (2007).
106. Zhang, L., Jiang, Y., Ding, Y., Povey, M. & York, D. Investigation into the antibacterial behaviour of suspensions of ZnO nanoparticles (ZnO nanofluids). Journal of Nanoparticle Research 9, 479-489 (2007).
107. Arciniegas-Grijalba, P. A., Patiño-Portela, M. C., Mosquera-Sanchez, L. P., Guerrero-Vargas, J. A. & Rodriguez-Piez, J. E. ZnO nanoparticles (ZnO-NPs) and their antifungal activity against coffee fungus Erythricium salmonicolor. Applied Nanoscience 7, 225-241 (2017).
108. Gondal, M. A., Alzahrani, A. J., Randhawa, M. A. & Siddiqui, M. N. Morphology and antifungal effect of nano-ZnO and nano-Pd-doped nano-ZnO against Aspergillus and Candida. Journal of Environmental Science and Health, Part A 47, 1413-1418 (2012).
109. Lipovsky, A., Nitzan, Y., Gedanken, A. & Lubart, R. Antifungal activity of ZnO nanoparticles—the role of ROS mediated cell injury. Nanotechnology 22, 105101 (2011).
110. Sharma, V. et al. DNA damaging potential of zinc oxide nanoparticles in human epidermal cells. Toxicology letters 185, 211-218 (2009).
111. Song, W. et al. Role of the dissolved zinc ion and reactive oxygen species in cytotoxicity of ZnO nanoparticles. Toxicology letters 199, 389-397 (2010).
112. Sharma, V., Anderson, D. & Dhawan, A. Zinc oxide nanoparticles induce oxidative DNA damage and ROS-triggered mitochondria mediated apoptosis in human liver cells (HepG2). Apoptosis 17, 852-870 (2012).
113. Arciniegas-Grijalba, P., Patiño-Portela, M., Mosquera-Sánchez, L., Guerrero-Vargas, J. & Rodriguez-Piez, J. ZnO nanoparticles (ZnO-NPs) and their antifungal activity against coffee fungus Erythricium salmonicolor. Applied Nanoscience 7, 225-241 (2017).
114. Sawangphruk, M., Srimuk, P., Chiochan, P., Sangsri, T. & Siwayaprahm, P. Synthesis and antifungal activity of reduced graphene oxide nanosheets. Carbon 50, 5156-5161 (2012).
115. Shahnawaz Khan, M., Abdelhamid, H. N. & Wu, H.-F. Near infrared (NIR) laser mediated surface activation of graphene oxide nanoflakes for efficient antibacterial, antifungal and wound healing treatment. Colloids and Surfaces B: Biointerfaces 127, 281-291 (2015).
116. Das, S. et al. Oxygenated functional group density on graphene oxide: its effect on cell toxicity. Particle & Particle Systems Characterization 30, 148-157 (2013).
117. Liao, K.-H., Lin, Y.-S., Macosko, C. W. & Haynes, C. L. Cytotoxicity of Graphene Oxide and Graphene in Human Erythrocytes and Skin Fibroblasts. ACS Applied Materials & Interfaces 3, 2607-2615 (2011).
118. Brunet, L.n., Lyon, D. Y., Hotze, E. M., Alvarez, P. J. & Wiesner, M. R. Comparative photoactivity and antibacterial properties of C60 fullerenes and titanium dioxide nanoparticles. Environmental science & technology 43, 4355-4360 (2009).
119. Haghighi, F., Roudbar Mohammadi, S., Mohammadi, P., Hosseinkhani, S. & Shipour, R. Antifungal activity of TiO2 nanoparticles and EDTA on Candida albicans biofilms. Infection, Epidemiology and Microbiology 1, 33-38 (2013).
120. Darbari, S., Abdi, Y., Haghighi, F., Mohajerzadeh, S. & Haghighi, N. Investigating the antifungal activity of TiO2 nanoparticles deposited on branched carbon nanotube arrays. Journal of Physics D: Applied Physics 44, 245401 (2011).
121. Haghighi, N., Abdi, Y. & Haghighi, F. Light-induced antifungal activity of TiO2 nanoparticles/ZnO nanowires. Applied Surface Science 257, 10096-10100 (2011).
122. Kim, I.-S., Baek, M. & Choi, S.-J. Comparative Cytotoxicity of Al2O3, CeO2, TiO2 and ZnO Nanoparticles to Human Lung Cells. Journal of Nanoscience and Nanotechnology 10, 3453-3458 (2010).
123. Ahmad, N. S., Abdullah, N. & Yasin, F. M. Antifungal Activity of Titanium Dioxide Nanoparticles against Candida albicans. BioResources 14, 8866-8878 (2019).
124. Guo, M. Y. et al. Annealing-Induced Antibacterial Activity in TiO2 under Ambient Light. The Journal of Physical Chemistry C 121, 24060-24068 (2017).
125. Pallavicini, P. et al. Self-assembled monolayers of gold nanostars: a convenient tool for near-IR photothermal biofilm eradication. Chemical Communications 50, 1969-1971 (2014).
126. Teng, C. P. et al. Effective Targeted Photothermal Ablation of Multidrug Resistant Bacteria and Their Biofilms with NIR-Absorbing Gold Nanocrosses. Advanced healthcare materials 5, 2122-2130 (2016).
127. Wisplinghoff, H. et al. Nosocomial bloodstream infections in US hospitals: analysis of 24,179 cases from a prospective nationwide surveillance study. Clinical infectious diseases 39, 309-317 (2004).
128. Rifai, A. et al. Engineering the Interface: Nanodiamond Coating on 3D-Printed Titanium Promotes Mammalian Cell Growth and Inhibits Staphylococcus aureus Colonization. ACS Applied Materials & Interfaces 11, 24588-24597 (2019).
129. Boulos, L., Prévost, M., Barbeau, B., Coallier, J. & Desjardins, R. LIVE/DEAD® BacLight™: Application of a New Rapid Staining Method for Direct Enumeration of Viable and Total Bacteria in Drinking Water. J. Microbiol. Methods 37, 77-86 (1999).
130. Heydorn, A. et al. Quantification of biofilm structures by the novel computer program COMSTAT. Microbiology 146, 2395-2407 (2000).
131. Vorregaard, M. (Citeseer, 2008).

### Definitions

Whenever a range is given in the specification, for example, a temperature range, a time range, or concentration range, all intermediate ranges and subranges, as well as all individual values included in the ranges given are intended to be included in the disclosure. It will be understood that any subranges or individual values in a range or subrange that are included in the description herein can be excluded from the claims herein.

All definitions, as defined and used herein, should be understood to control over dictionary definitions, definitions in documents incorporated by reference, and/or ordinary meanings of the defined terms.

Throughout this application, the term “about” is used to indicate that a value includes the inherent variation of error for the device, the method being employed to determine the value, or the variation that exists among the study subjects.

The indefinite articles “a” and “an,” as used herein in the specification, unless clearly indicated to the contrary, should be understood to mean “at least one.”

The phrase “and/or,” as used herein in the specification, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the Figures. Spatially relative terms may be intended to encompass different orientations of the device in use or operation in addition to the orientation depicted in the Figures.

The term “ambient conditions” is to be understood depending on the context in which it is used. When used in the context of assessing the antimicrobial efficacy of a coating “ambient conditions” refers to standard cell culture conditions of 37° C., 5% CO₂ and 95% relative humidity.

While the invention has been described in conjunction with a limited number of embodiments, it will be appreciated by those skilled in the art that many alternatives, modifications and variations in light of the foregoing description are possible. Accordingly, the present invention is intended to embrace all such alternatives, modifications and variations as may fall within the spirit and scope of the invention as disclosed.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

1. An article comprising a substrate and an anti-microbial coating, wherein the anti-microbial coating comprises at least one solvent-free black phosphorus flake.

2. An article according to claim 2, wherein the at least one black phosphorus flake generates reactive oxygen species (ROS) that are active towards at least some types of micro-organisms.

3. An article according to claim 1 or 2, wherein the anti-microbial coating prevents the growth of, or kills, one or more micro-organisms selected from the group consisting of bacterial cells and fungal cells or spores.

4. An article according to any one of claims 1 to 3, wherein the anti-microbial coating has a density that falls within a range of from about 0.1 ng of black phosphorus per μm² of substrate to about 1.0 ng of black phosphorus per μm² of substrate.

5. An article according to any one of claims 1 to 4, wherein the anti-microbial coating has a density that falls within a range of from about 0.2 ng of black phosphorus per μm² of substrate to about 0.6 ng of black phosphorus per μm² of substrate.

6. An article according to any one of claims 1 to 4, wherein the anti-microbial coating has a density of about 0.4 ng of black phosphorus per μm² of substrate.

7. An article according to any one of claims 1 to 6, wherein the anti-microbial coating prevents the growth of, or kills, one or more bacterial species selected from the group consisting of: Escherichia coli, Pseudomonas aeruginosa, methicillin-resistant Staphylococcus aureus (MRSA) (resistive species), Salmonella typhimurium, or Bacillus cereus.

8. An article according to any one of claims 1 to 6, wherein the anti-microbial coating prevents the growth of, or kills, one or more fungal species selected from the group consisting of: Candida albicans, Candida auris (C. auris), sensitive Cryptococcus neoformans, fluconazole-resistant Cryptococcus neoformans, or Ampicillin-resistant Cryptococcus neoformans.

9. An article according to any one of claims 1 to 6, wherein the anti-microbial coating kills at least about 90% of microorganisms within a period of 120 minutes under ambient conditions, wherein the microorganism is selected from the group consisting of: methicillin-resistant Staphylococcus aureus, or Escherichia coli, or Pseudomonas aeruginosa, or Salmonella typhimurium, or Bacillus cereus, or Candida albicans, or Candida auris, or Sensitive Cryptococcus neoformans, or fluconazole-resistant Cryptococcus neoformans, or Ampicillin-resistant Cryptococcus neoformans.

10. An article according to any one of claims 1 to 9, wherein the at least one black phosphorus flake has an average thickness that falls within a range of from about 10 nm to about 120 nm.

11. An article according to any one of claims 1 to 9, wherein the at least one black phosphorus flake has an average thickness that falls within a range of from about 15 nm to about 90 nm.

12. An article according to any one of claims 1 to 11, wherein the at least one black phosphorus flake has an average lateral dimension that falls within a range of from about 500 nm to about 5 μm.

13. An article according to any one of claims 1 to 12, wherein the at least one black phosphorus flake is produced from a black phosphorus crystal by mechanical exfoliation.

14. An article according to claim 13, wherein the mechanical exfoliation is conducted in a solvent-free environment.

15. An article according to any one of claims 1 to 14, wherein the at least one black phosphorus flake is physically adsorbed on to a surface of the substrate.

16. An article according to any one of claims 1 to 14, wherein the at least one black phosphorus flake is deposited onto a surface of the substrate by contacting the substrate surface with an applicator having at least one black phosphorus flake in contact with a surface thereof.

17. An article according to any one of claims 2 to 16, wherein the reactive oxygen species (ROS) are generated when the black phosphorus flake is exposed to atmospheric oxygen.

18. An article according to any one of claims 2 to 17, wherein the reactive oxygen species (ROS) are generated under ambient conditions.

19. An article according to any one of claims 2 to 18, wherein the reactive oxygen species are selected from the group consisting of a singlet oxygen radical (¹O₂), a hydroxy radical (OH.), a superoxide radical (O₂.⁻) and hydrogen peroxide (H₂O₂).

20. An article according to any one of claims 2 to 19, wherein the reactive oxygen species (ROS) are not substantially cytotoxic towards mammalian cells.

21. An article according to any one of claims 1 to 20, wherein the article is an implant, a medical instrument, a bioscaffold, or a woven or non-woven textile.

22. An article according to any one of claims 1 to 21, wherein the substrate is selected from the group consisting of: a metal, an alloy, a polymer, a textile, a glass, a ceramic, or any combination thereof.

23. An article according to claim 22, wherein the metal is selected from the group consisting of titanium, gold, stainless steel, aluminium, copper, or any combination thereof.

24. An article according to any one of claims 1 to 23, wherein the at least one black phosphorous flake is adhered to the substrate.

25. A method of producing an article having an anti-microbial coating, comprising:
providing a substrate;
depositing at least one black phosphorus flake onto a surface of the substrate in the absence of solvent.

26. A method according to claim 25, wherein the at least one black phosphorus flake generates reactive oxygen species (ROS) that are active towards at least some types of micro-organisms.

27. A method according to claim 25 or 26, wherein the anti-microbial coating prevents the growth of, or kills, micro-organisms selected from the group consisting of bacterial cells, and fungal cells or spores.

28. A method according to any one of claims 25 to 27, wherein the anti-microbial coating has a density that falls within a range of from about 0.1 ng of black phosphorus per μm² of substrate to about 1.0 ng of black phosphorus per μm² of substrate.

29. A method according to any one of claims 25 to 28, wherein the anti-microbial coating has a density that falls within a range of from about 0.2 ng of black phosphorus per μm² of substrate to about 0.6 ng of black phosphorus per μm² of substrate.

30. A method according to any one of claims 25 to 28, wherein the anti-microbial coating has a density that falls within a range of from about 0.4 ng of black phosphorus per μm² of substrate.

31. A method according to any one of claims 25 to 30, wherein the anti-microbial coating prevents the growth of, or kills, one or more bacterial species selected from the group consisting of: Escherichia coli, Pseudomonas aeruginosa, methicillin-resistant Staphylococcus aureus (MRSA) (resistive species), Salmonella typhimurium, or Bacillus cereus.

32. A method according to any one of claims 25 to 30, wherein the anti-microbial coating prevents the growth of, or kills, one or more fungal species selected from the group consisting of: Candida albicans, sensitive Cryptococcus neoformans, fluconazole-resistant Cryptococcus neoformans, or Ampicillin-resistant Cryptococcus neoformans.

33. A method according to any one of claims 25 to 30, wherein the anti-microbial coating kills at least about 90% of microorganisms within a period of 120 minutes under ambient conditions, wherein the microorganism is selected from the group consisting of: methicillin-resistant Staphylococcus aureus, or Escherichia coli, or Pseudomonas aeruginosa, or Salmonella typhimurium, or Bacillus cereus, or Candida albicans, or Candida auris, or Sensitive Cryptococcus neoformans, or fluconazole-resistant Cryptococcus neoformans, or Ampicillin-resistant Cryptococcus neoformans.

34. A method according to any one of claims 25 to 33, wherein the at least one black phosphorus flake has an average thickness that falls within a range of from about 10 nm to about 120 nm.

35. A method according to any one of claims 25 to 33, wherein the at least one black phosphorus flake has an average thickness that falls within a range of from about 15 nm to about 90 nm.

36. A method according to any one of claims 25 to 35, wherein the at least one black phosphorus flake has an average lateral dimension that falls within a range of from about 500 nm to about 5 μm.

37. A method according to any one of claims 25 to 36, wherein the at least one black phosphorus flake is produced from a black phosphorus crystal by mechanical exfoliation.

38. A method according to claim 37, wherein the mechanical exfoliation is conducted in a solvent-free environment.

39. A method according to any one of claims 25 to 38, wherein the at least one black phosphorus flake is physically adsorbed on to a surface of the substrate.

40. A method according to any one of claims 25 to 38, wherein the at least one black phosphorus flake is deposited onto a surface of the substrate by contacting the substrate surface with an applicator having at least one black phosphorus flake in contact with a surface thereof.

41. A method according to any one of claims 26 to 40, wherein the reactive oxygen species (ROS) are generated when the black phosphorus flake is exposed to atmospheric oxygen.

42. A method according to any one of claims 26 to 41, wherein the reactive oxygen species (ROS) are generated under ambient conditions.

43. A method according to any one of claims 26 to 42, wherein the reactive oxygen species are selected from the group consisting of a singlet oxygen radical (¹O₂), a hydroxy radical (OH.), a superoxide radical (O₂.⁻) and hydrogen peroxide (H₂O₂).

44. A method according to any one of claims 26 to 43, wherein the reactive oxygen species (ROS) are not substantially cytotoxic towards mammalian cells.

45. A method according to any one of claims 25 to 44, wherein the at least one black phosphorous flake is adhered to the substrate.

46. A method according to any one of claims 25 to 45, wherein the substrate is selected from the group consisting of: a metal, an alloy, a polymer, a textile, a glass, a ceramic, or any combination thereof.

47. A method according to claim 46, wherein the metal is selected from the group consisting of titanium, gold, stainless steel, aluminium, copper, or any combination thereof.

48. A method according to any one of claims 25 to 47, wherein the substrate is produced from a medical grade metal or alloy.

49. A method according to any one of claims 25 to 47, wherein the substrate is a surgical implant.

50. A method according to any one of claims 25 to 47, wherein the substrate is a bioscaffold.

51. A method according to any one of claims 25 to 47, wherein the substrate is a woven or non-woven textile.

52. An article produced according to the method of any one of claims 25 to 51.

53. Use of at least one solvent-free black phosphorus flake in the manufacture of an anti-microbial coating for a substrate.

54. A solvent-free black phosphorous flake for use in an anti-microbial coating on a substrate.

55. A method of producing at least one solvent-free black phosphorus flake with anti-microbial activity, comprising:
contacting a black phosphorus crystal with an applicator having an adhesive surface to cause the applicator to adhere to a surface of the black phosphorus crystal; and
removing the applicator from the surface of the black phosphorous crystal, thereby mechanically exfoliating at least one black phosphorous flake from the black phosphorous crystal.

56. An anti-microbial coating, comprising:
at least one black phosphorus flake produced in the absence of solvent.

57. An anti-microbial coating according to claim 56, wherein the at least one black phosphorus flake generates reactive oxygen species (ROS) that are active towards at least some types of micro-organisms.
An article comprising a substrate and an anti-microbial coating according to claim 56 or 57, wherein the at least one black phosphorus flake is deposited on a surface of the substrate in the absence of solvent.
